(19) United States
(12) Patent Application Publication
(10) Pub. No.: US 2026/0090991 A1
(43) Pub. Date: Apr. 2, 2026

(54) COMPOSITIONS AND METHODS OF DELIVERING LARGE PROTEINS

(71) Applicant: BROWN UNIVERSITY, Providence, RI (US)

(72) Inventors: Edith MATHIOWITZ, Providence, RI (US); Aharon AZAGURY, Kiryat Gat (IL); Cameron BAPTISTA, Pittsburgh, PA (US)

(21) Appl. No.: 19/115,269

(22) PCT Filed: Oct. 3, 2023
(86) PCT No.: PCT/US2023/075810

§ 371 Date: Mar. 26, 2025

Related U.S. Application Data
(60) Provisional application No. 63/378,318, filed on Oct. 4, 2022.

(51) Int. Cl. A61K 9/16; A61K 9/00; C07K 16/28

(52) U.S. Cl. CPC A61K 9/1647; A61K 9/0019; C07K 16/2839; C07K 2317/90

(57) ABSTRACT

Disclosed are pharmaceutical compositions containing micronized antibodies encapsulated and/or dispersed in polymeric particles for antibody delivery. The judicious identification of (i) a subset of polymers, (ii) polymers with certain average molecular weights, (iii) a subset of antibody loadings, and/or (iv) pre-loading antibody processing, leads to formation of polymeric particles that possess minimal to no initial burst release of micronized antibody at zero time point. The pharmaceutical compositions are formulated for oral, subcutaneous, or percutaneous administration, and are particularly suited for treatment regimens that involve antibody-based therapy.

### CROSS-REFERENCE TO RELATED APPLICATIONS

This application claims benefit and priority to U.S. Application No. 63/378,318, filed Oct. 4, 2022, which is incorporated herein by reference in its entirety.

### FIELD OF THE INVENTION

The disclosed invention is generally in the field of delivery of therapeutic, diagnostic, and/or prophylactic agents, particularly oral, subcutaneous, percutaneous, and/or mucosal administration of antibodies in antibody-based therapies.

### BACKGROUND OF THE INVENTION

The use of microparticles and nanoparticles in pharmaceutical compositions to deliver drugs and other bioactive agents is well known in the art. Polymeric microparticles or nanoparticle systems have great potential for drug delivery systems due to their ability to shield active agents from external solvents and degradants. Such systems are especially useful in the context of oral drug delivery due to their ability to protect active agents from the harsh gastrointestinal tract. Although significant effort has focused on developing effective delivery systems for clinical use, significant obstacles exist in the development of effective systems for biologics, particularly high molecular weight proteins, such as antibodies.

Because of their high molecular weight and susceptibility to degradation by both enzymes and extreme pH values, antibodies usually display poor absorption across epithelial membranes and exhibit low oral bioavailability. Further, certain particle sizes, types of polymer, and/or antibody stabilizing excipients lead to significant and undesirable initial burst release kinetics in polymeric microparticles or nanoparticles. Accordingly, there exists a need for improved pharmaceutical compositions for delivering antibodies, particularly in clinical settings involving antibody-based therapies.

Therefore, it is an object of the invention to provide improved pharmaceutical compositions for delivering antibodies.

It is a further object of the invention to provide improved pharmaceutical compositions for delivering antibodies, which possess minimal to no burst release of the antibody at zero time point.

It is a further object of the invention to provide improved pharmaceutical compositions for delivering antibodies, the pharmaceutical compositions containing micronized antibodies encapsulated and/or dispersed in polymeric microparticles, nanoparticles, or a combination thereof, wherein minimal to no burst release of the micronized antibody occurs at zero time point.

### BRIEF SUMMARY OF THE INVENTION

Disclosed are pharmaceutical compositions for antibody delivery, with minimal to no burst release of the antibody in a micronized form at zero time point. The pharmaceutical compositions contain a micronized antibody having a molecular weight of 100 kDa or greater, encapsulated and/or dispersed in a microparticle, a nanoparticle, or a combination thereof. The micronized antibody has a diameter between at least 20 nm and less than 600 nm, and has a loading between 0.3% w/w and 15% w/w. In some forms, the micronized antibody is composed of less than 50% w/w antibody stabilizing excipients having a molecular weight of less than 20 kDa. This can be achieved through dialyzation of the antibody using a 20-kDa molecular weight cut off dialysis cassette prior to micronization to remove antibody stabilizing excipient.

The particles contain a polyhydroxyester polymer having an average molecular weight between about 2 kDa and about 30 kDa. In some forms, the particles contain a blend of two or more polyhydroxyester polymers, including at least a polyhydroxyester homopolymer. Preferably, the particles contain a polylactic acid polymer alone or in a blend with poly(lactic acid-co-glycolic acid). The nanoparticles can have a size between at least 50 nm and less than 1 μm, while the microparticles can have a size between at least 1 μm and about 2 μm.

Also disclosed are methods of using the pharmaceutical compositions. The pharmaceutical compositions are particularly suited for treatment regimens that involve antibody-based therapy. Preferably, the pharmaceutical compositions are formulated for oral, subcutaneous, percutaneous, or mucosal administration of a micronized antibody encapsulated or dispersed in the particles.

Also disclosed are methods of making a micronized antibody having a molecular weight of 100 kDa or greater, encapsulated and/or dispersed a microparticle, a nanoparticle, or a combination thereof. A preferred method is phase inversion nanoencapsulation.

### BRIEF DESCRIPTION OF THE DRAWINGS

FIG. 1 is a line graph showing the release profile data. Percent release of encapsulated drug over time (˜24 hours) for formulations FD1, FD2, F19, F20, F21, F34, F38, F39, F40 containing non-dialyzed drug and F54 containing the dialyzed drug is shown.

FIG. 2 is a line graph showing the release profile data. Percent release of encapsulated drug over time (˜24 hours) for formulations F25, F28, F42 and F48 containing non-dialyzed drug and F52 containing the dialyzed drug is shown.

FIG. 3 is line graph showing the release profile data. Percent release of encapsulated drug over time (˜24 hours) for formulations FD5, FD6, F24 and F32, F33, and F41 containing non-dialyzed drug is shown.

FIG. 4 is a line graph showing the release profile data. Percent release of encapsulated drug over time (˜24 hours) for formulations FD9 and FD10, containing non-dialyzed drug is shown.

FIG. 5 is a line graph showing the release profile data. Percent release of encapsulated drug over time (˜24 hours) for formulations FD1, FD5, FD9, F20, F24, F25, F32, and F33 containing non-dialyzed drug is shown.

FIG. 6 is a line graph showing the release profile data for formulations containing dialyzed drug. Percent release of encapsulated drug over time (˜1 week) for formulations F69, F75, F77, F78, and F82 comprising only one polymer is shown.

FIG. 7 is a line graph showing the release profile data over time for formulations containing dialyzed drug. Percent release of encapsulated drug over one week for formulations F55, F56, F73 and F75 is shown.

FIG. 8 is a line graph illustrating the effects of different dialyzed drug loadings on the release of drug encapsulated by a single polymer or combinations of polymers.

FIGS. 9A and 9B are line graphs showing the release profiles of formulations before (FIG. 9A) and after (FIG. 9B) dialysis. FIG. 9A shows burst release with a significant number of the formulations having zero time point release above 10%. FIG. 9B shows burst release at the zero time point is considerably decreased after dialysis. In general, after six hours, the slopes of the cumulative release curves for the formulations in (FIG. 9B) have not plateaued (compared to those in FIG. 9A), which indicates a gradual micronized antibody release over long periods of time.

FIGS. 10A-10D are line graphs showing the release profiles of representative formulations after dialysis. Burst release at the zero time point is considerably suppressed, and all formulations show reliable release kinetics.

FIG. 11 is a line graph showing different ratios of polylactic acid polymer (PLA) and poly(lactic acid-co-glycolic acid) polymer (PLGA) in varying combinations of molecular weights and lactic acid to glycolic acid ratios that can be used to control release dynamics.

FIG. 12 is a line graph showing different ratios of PLGA in varying combinations of molecular weights and lactic acid to glycolic acid ratios that can be used to control release dynamics. a: F60-PLLA 2 kDa-DL 4.2%; b: F66-PLLA 2 kDa+PLGA (85:15) 10 kDA+PLGA (50:50) 4 kDa-25:40:35; c: F65-PLLA 2 kDa+PLGA (85:15) 10 kDA+PLGA (50:50) 4 kDa-25:50:25; d: F64-PLLA 2 kDa+PLGA (85:15) 10 kDA+PLGA (50:50) 4 kDa-25:60:15.

FIG. 13 is a line graph showing the serum concentrations (μL/mL) detected over time following intravenous injection of the antibody.

FIG. 14 is a line graph showing the serum concentrations (μL/mL) detected over time following subcutaneous injection of the antibody.

### DETAILED DESCRIPTION OF THE INVENTION

The disclosed method and compositions can be understood more readily by reference to the following detailed description of particular embodiments and the Example included therein and to the Figures and their previous and following description.

### I. Definitions

“Antibody stabilizing excipient,” refers to non-polymeric molecules that are commonly used to stabilize an antibody in an aqueous buffer. The antibody stabilizing excipient typically has a molecular weight of less than 20 kDa. The type of antibody stabilizing excipient depends on the type of antibody, and can be amino acids, such as histidine and arginine. The antibody stabilizing excipient can also be water-soluble.

### II. Pharmaceutical Compositions

Provided are pharmaceutical compositions for delivering an antibody in micronized form dispersed and/or encapsulated in particles. After several experiments, it has been discovered that particles (i) containing a subset of polymers, (ii) containing polymers with certain average molecular weights, (iii) having a subset of antibody loadings, and/or (iv) dialyzed antibody, give rise to antibody release kinetics with minimal to no initial burst release (i.e., less than 10% w/w of micronized antibody is releases at zero time point). In some forms, it is the combination of (i) and (ii) that gives rise to minimal to no initial burst release. In some forms, it is the combination of (i) and (iv) that gives rise to minimal to no initial burst release. In some forms, it is the combination of (i), (ii), and (iii) that gives rise to minimal to no initial burst release. In some forms, it is the combination of (i), (ii), and (iv) that gives rise to minimal to no initial burst release. In some forms, it is the combination of (i), (ii), (iii), and (iv) that gives rise to minimal to no initial burst release.

Unless stated otherwise, references to the weight percent of each component in the nanoparticles, microparticles, or combination thereof provided herein refer to the weight of the component divided by the total weight of all of the components in the nanoparticles, microparticles or combination thereof. Thus, it is understood that the sum of the weight percentages of all of the components in the nanoparticles, microparticles or combination thereof is 100% w/w.

The pharmaceutical compositions contain a micronized antibody having a molecular weight of 100 kDa or greater, encapsulated and/or dispersed in particles selected from a microparticle, a nanoparticle, or a combination thereof. Preferably, the micronized antibody has a diameter between at least 20 nm and less than 600 nm, and the nanoparticles, microparticles, or combination thereof contain micronized antibody in an amount of between 0.3% w/w and 15% w/w.

Preferably, the particles contain a polyhydroxyester polymer (such as a polyhydroxyester homopolymer) having an average molecular weight between about 2 kDa and about 30 kDa. In some forms, the particles contain a blend of two or more polyhydroxyester polymers, including at least a polyhydroxyester homopolymer. Preferably, the particles have a size between at least 50 nm and about 2 μm. For instance, the nanoparticles can have a size between at least 50 nm and less than 1 μm, while the microparticles can have a size between at least 1 μm and about 2 μm.

Preferably, the pharmaceutical composition is formulated for oral, subcutaneous, percutaneous, or mucosal administration of a micronized antibody encapsulated and/or dispersed in the particles.

Further details about the disclosed pharmaceutical compositions are provided in the ensuing paragraphs.

### 1. Microparticles and Nanoparticles

The particles described herein generally are in the form of microparticles, nanoparticles, or a combination thereof.

i. Microparticle or Nanoparticle Size

The disclosed microparticles, nanoparticles, or combinations thereof, typically have a size between about 50 nm to about 50 μm, inclusive, such as between about 0.3 μm to about 50 μm. Based upon whether delivery is local, such as to the GI tract, or to the systemic circulation, the particles may have different size sub-ranges within this range. Typically, the sizes of the particles for local delivery are in the range from about 2 μm to about 10 μm. Typically, the diameters of the particles for systemic delivery are in the range from about 0.3 μm to about 2 μm.

The microparticles can be microspheres, microcapsules, and/or structures that may not be readily placed into either of the above two categories, all with mean particle sizes of less than about 1000 microns. The microparticles may be microspheres that are substantially spherical colloidal structures formed from polyhydroxyester polymers and having a size ranging from about 1 micron or greater up to about 1000 microns. In some forms, the microparticle has a size of between at least 1 μm and about 50 μm, between at least 1 μm and about 40 μm, between at least 1 μm and about 30 μm, between at least 1 μm and about 20 μm, between at least 1 μm and about 10 μm, between at least 1 μm and about 5 μm, or between at least 1 μm and about 2 μm.

Nanoparticles have a mean particle size of less than one micron, and include nanospheres, and nanocapsules. In some forms, the nanoparticle has a size of between at least 50 nm and less than 1 μm, between at least 100 nm and less than 1 μm, between at least 200 nm and less than 1 μm, between at least 300 nm and less than 1 μm, between at least 400 nm and less than 1 μm, between at least 500 nm and less than 1 μm, or between at least 600 nm and less than 1 μm. In certain forms, the nanoparticles have a mean particle size of about 500 nm, 200 nm, 100 nm, or 50 nm, or 10 nm.

Mean particle size generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. The diameter of a non-spherical particle may refer to the largest linear distance between two points on the surface of the particle. Mean particle size can be measured using methods known in the art, such as dynamic light scattering.

ii. Polymers

The disclosed pharmaceutical compositions may contain a microparticle, a nanoparticle, or a combination thereof, formed of a single polymer. Preferably, the polymer is a polyhydroxyester homopolymer, such as poly-L-lactic acid (PLLA) or poly-D-lactic acid (PDLA). In some forms, the microparticle, nanoparticle, or combination thereof is formed of a blend containing two or more polymers. Preferably, the blend contains (i) a polyhydroxyester homopolymer (such as PLLA and/or PDLA) and a polyhydroxyester co-polymer (such as PLGA, PDLLA, etc) or (ii) two or more polyhydroxyester homopolymers, such as a blend between PLLA and PLDA, PLLA and PDLLA, or PLDA and PDLLA.

a. Exemplary Polymers

Preferably, the polymers for forming the particles of the pharmaceutical composition are biocompatible, biodegradable, or a combination thereof.

Biodegradable polymers can be used as the polymer for drug delivery applications, wherein one or more encapsulated active agents are released over time as the polymer degrades.

The microparticles and nanoparticles typically contain at least one or more biodegradable polyesters. Preferably, the biodegradable polyester is a polyhydroxyester polymer. Preferably, the particles contain one or more of the following polyhydroxyesters: homopolymers including glycolic acid units, referred to herein as “PGA”; and lactic acid units, such as poly-L-lactic acid, poly-D-lactic acid, poly-L-lactide, poly-D-lactide, and poly-D,L-lactide, collectively referred to herein as “PLA”, and copolymers such as those including D-lactic acid units and L-lactic acid units, called poly-D,L-lactic acid; or those including lactic acid and glycolic acid units, such as various forms of poly(lactic acid-co-glycolic acid) and poly(lactide-co-glycolide) characterized by the ratio of lactic acid to glycolic acid, collectively referred to herein as “PLGA”. In preferred forms, the antibodies (such as micronized antibodies) are complexed with a polyhydroxyester polymer such as poly-L-lactide acid (“PLLA”), poly-D-lactide acid (“PDLA”), poly-D,L-lactide, and poly(lactic-co-glycolic acid) (PLGA), and combinations thereof to form a particle with micrometer or nanometer dimensions. The following pairs of phrases are used interchangeably: poly-L-lactic acid and poly-L-lactide; poly-D-lactic acid and poly-D-lactide; poly-D,L-lactide and poly-D,L-lactic acid; and poly(lactide-co-glycolide) and poly(lactic-co-glycolic acid).

Other examples of biodegradable polyesters include, but are not limited to aliphatic polyesters such as poly(caprolactone), poly(butylene succinate), poly(p-dioxanone), polycarbonate; and aromatic copolyesters such as poly(butylene adipate-co-terephthalate, and copolymers of terephthalic acid and hydroxy acids such as PLA and/or PLGA.

Optionally, the composition contains one or more bioadhesive polymers or is coated with one or more bioadhesive polymers. Suitable bioadhesive polymers are described in the Optional Features section below.

b. Molecular Weights of the Polyhydroxyester Polymers

The average molecular weight range for the polyhydroxyester polymer in the particles can range from about 1 kDa to about 200 kDa, about 2 kDa to about 200 kDa, about 2 kDa to about 150 kDa, about 2 kDa to about 110 kDa, about 2 kDa to about 100 kDa, about 2 to about 60 kDa, about 1 kDa to about 120 kDa, about 4 kDa to about 14 kDa, about 6 to about 14 kDa, about 1 kDa to about 25 kDa, about 1.5 kDa to about 15 kDa, about 2 kDa to about 12 kDa, or about 2 kDa to about 11 kDa. In some forms, one or more polyhydroxyester polymers, or all of the polyhydroxyester polymers, forming the particles have an average molecular weight of about 1 kDa, 2, kDa, 3 kDa, 4 kDa, 5 kDa, 6 kDa, 7 kDa, 8 kDa, 9 kDa, 10 kDa, 11 kDa, 12 kDa, 13 kDa or 14 kDa.

In some forms, the microparticle, nanoparticle, or combination thereof contains a poly-L-lactide (PLLA) polymer. In some forms, the average molecular weight for the PLLA polymer is about 10 kDa or less. Preferably, the average molecular weight for the PLLA polymer is about 2 kDa, about 5 kDa, or about 10 kDa. Preferably, the PLLA polymer constitutes at least about 10% w/w of the polyhydroxyester polymer(s). In some forms, preferably, the PLLA polymer constitutes about 10% w/w of the microparticle, nanoparticle, or combination thereof.

In some forms, the microparticle, nanoparticle, or combination thereof contains a poly-D-lactide (PDLA) polymer. Preferably, the average molecular weight for the PDLA polymer ranges from about 2 kDa to about 14 kDa. In some forms, the PDLA polymer constitutes about 10% w/w of the polyhydroxyester polymer(s). In some forms, the PDLA polymer constitutes about 10% w/w of the microparticle, nanoparticle, or combination thereof.

In some forms, the microparticle, nanoparticle, or combination thereof contains a poly(lactic acid-co-glycolic acid (PLGA) polymer. Preferably, the average molecular weight for the PLGA polymer ranges from about 1 kDa to about 120 kDa, about 2 kDa to about 110 kDa, about 2 kDa to about 60 kDa, about 4 kDa to about 14 kDa, or, more preferably, from about 6 kDa to about 14 kDa. In some forms, the PLGA polymers contain a lactic acid: glycolic acid molar ratio of 85% lactic acid to 15% glycolic acid or 50% lactic acid to 50% glycolic acid.

In some forms, the microparticle, nanoparticle, or combination thereof contains at least two of the polyhydroxyester polymers (i), (ii), and (iii):
(i) about 10% w/w to about 80% w/w PLLA of about 1 kDa to about 10 kDa or about 2 kDa to about 10 kDa;
(ii) about 10% w/w to about 80% w/w PDLA of about 1 kDa to about 10 kDa or about 2 kDa to about 10 kDa; and
(iii) about 10% w/w to about 80% w/w PLGA of about 1 kDa to about 120 kDa.

In some forms, the microparticle, nanoparticle, or combination thereof contains at least two of the polyhydroxyester polymers (i), (ii), and (iii):
(i) about 10% w/w to about 80% w/w PLLA of about 1 kDa to about 10 kDa or about 2 kDa to about 10 kDa;
(ii) about 10% w/w to about 80% w/w PDLA of about 1 kDa to about 10 kDa or about 2 kDa to about 10 kDa; and
(iii) about 10% w/w to about 80% w/w PLGA of about 1 kDa to about 120 kDa,
wherein the percent weights of the polymers are the polymer weight ratios between the polyhydroxyester polymers. For example, in these forms, the weight ratio is polyhydroxyester (i):polyhydroxyester (ii); polyhydroxyester (i):polyhydroxyester (iii); polyhydroxyester (ii):polyhydroxyester (iii); or polyhydroxyester (i):polyhydroxyester (ii):polyhydroxyester (iii).

In some forms, the microparticle, nanoparticle, or combination thereof contains:
(i) about 35% w/w to about 80% w/w, about 50% w/w to about 80% w/w, or about 55 w/w to about 80% w/w PLLA of about 1 kDa to about 10 kDa or about 1 kDa to about 5 kDa, or about 2 kDa; and
(ii) about 20% w/w to 50% w/w, or about 20% w/w to about 45% w/w of about 1 kDa to about 120 kDa, about 4 kDa, about 10 kDa, about 38-54 kDa, or about 110 kDa PLGA, preferably wherein lactide and glycolide residues in the PLGA are present in a ratio of about 50:50 or about 85:15.

In some forms, the microparticle, nanoparticle, or combination thereof contains:
(i) about 35% w/w to about 80% w/w, about 50% w/w to about 80% w/w, or about 55 w/w to about 80% w/w PLLA of about 1 kDa to about 10 kDa or about 1 kDa to about 5 kDa, or about 2 kDa; and
(ii) about 20% w/w to 50% w/w, or about 20% w/w to about 45% w/w of about 1 kDa to about 120 kDa, about 4 kDa, about 10 kDa, about 38-54 kDa, or about 110 kDa PLGA, preferably wherein lactide and glycolide residues in the PLGA are present in a ratio of about 50:50 or about 85:15,
wherein the percent weights of the polymers are the polymer weight ratios between the polyhydroxyester polymers. For example, in these forms, the weight ratio is polyhydroxyester (i):polyhydroxyester (ii).

In some forms, the microparticle, nanoparticle, or combination thereof contains at least two of the polyhydroxyester polymers (i), (ii), and (iii):
(i) 1-30% w/w PLLA of 2-10 kDa;
(ii) 1-50% w/w PDLA of 2-10 kDa;
(iii) 70-96% w/w PLGA of 2-60 kDa.

In some forms, the microparticle, nanoparticle, or combination thereof contains at least two of the polyhydroxyester polymers (i), (ii), and (iii):
(i) 1-30% w/w PLLA of 2-10 kDa;
(ii) 1-50% w/w PDLA of 2-10 kDa;
(iii) 70-96% w/w PLGA of 2-60 kDa,
wherein the percent weights of the polymers are the polymer weight ratios between the polyhydroxyester polymers. For example, in these forms, the weight ratio is polyhydroxyester (i):polyhydroxyester (ii); polyhydroxyester (i):polyhydroxyester (iii); polyhydroxyester (ii):polyhydroxyester (iii); or polyhydroxyester (i):polyhydroxyester (ii):polyhydroxyester (iii).

In some forms, the microparticle, nanoparticle, or combination thereof contains at least two of the polyhydroxyester polymers (i), (ii), (iii), and (iv):
(i) 1-50% w/w PLLA of 2-10 kDa;
(ii) 1-50% w/w PDLA of 2-10 kDa
(iii) 1-70% w/w PLGA (85:15) of 2-60 kDa;
(iv) 0-30% w/w PLGA (50:50) of 2-60 kDa.

In some forms, the microparticle, nanoparticle, or combination thereof contains at least two of the polyhydroxyester polymers (i), (ii), (iii), and (iv):
(i) 1-50% w/w PLLA of 2-10 kDa;
(ii) 1-50% w/w PDLA of 2-10 kDa
(iii) 1-70% w/w PLGA (85:15) of 2-60 kDa;
(iv) 0-30% w/w PLGA (50:50) of 2-60 kDa,
wherein the percent weights of the polymers are the polymer weight ratios between the polyhydroxyester polymers. For example, in these forms, the weight ratio is polyhydroxyester (i):polyhydroxyester (ii); polyhydroxyester (i):polyhydroxyester (iii); polyhydroxyester (i):polyhydroxyester (iv); polyhydroxyester (ii):polyhydroxyester (iii); polyhydroxyester (ii):polyhydroxyester (iv), polyhydroxyester (iii):polyhydroxyester (iv), polyhydroxyester (i):polyhydroxyester (ii):polyhydroxyester (iii), or combinations selected from polyhydroxyester (i), (ii), (iii) and (iv).

In some forms, the microparticle, nanoparticle, or combination thereof contains:
(i) 50-100% w/w PLLA of 2-20 kDa;
(ii) 0-35% w/w PLGA (85:15) of 10-50 kDa. Preferably, in these forms, the pharmaceutical composition is suitable for subcutaneous or intraperitoneal injection.

In some forms, the microparticle, nanoparticle, or combination thereof contains:
(i) 50-100% w/w PLLA of 2-20 kDa;
(ii) 0-35% w/w PLGA (85:15) of 10-50 kDa,
wherein the percent weights of the polymers are the polymer weight ratios between the polyhydroxyester polymers. For example, in these forms, the weight ratio is polyhydroxyester (i):polyhydroxyester (ii). Preferably, in these forms, the pharmaceutical composition is suitable for subcutaneous or intraperitoneal injection.

Suitable polymer concentrations in the polymer solution range from about 0.01 to about 50% (weight/volume), depending primarily upon the molecular weight of the polymer and the resulting viscosity of the polymer solution. In general, the low molecular weight polymers permit usage of higher polymer concentrations. The preferred concentration range is from about 0.1% to about 20% (weight/volume), optionally the concentration is about 10% (weight/volume) or lower. Polymer concentrations ranging from about 1% to about 10% (weight/volume) or from about 1% to about 5% (weight/volume) are particularly useful for the compositions described herein.

The viscosity of the polymer solution preferably is less than about 3.5 centipoise and more preferably less than about 2 centipoise, although higher viscosities such as about 4 or even about 6 centipoise are possible depending upon adjustment of other parameters such as molecular weight.

It will be appreciated by those of ordinary skill in the art that polymer concentration, polymer molecular weight, and viscosity are interrelated, and that varying one will likely affect the others.

### 2. Active Agents

The disclosed particles contain an antibody, optionally more than one antibody. Optionally, in addition to one or more antibodies, the particles contain an active agent or more than one active agent that is not an antibody. The active agent or each active agent of two or more active agents dispersed and/or encapsulated within the particle can be a therapeutic agent, a diagnostic agent, or a prophylactic agent. The active agent(s) is/are delivered to the blood stream (systemic) or to the GI tract or other mucosal surfaces, such as vagina (local) in a mammal via the particles. Upon reaching their target locally or systemically, the particles containing the active agent(s) can break down to release the active agent(s) in a controlled manner.

The antibodies are large proteins with an average molecular weight of about 100 kDa or greater. For example, in some forms, the antibody has an average molecular weight of at least 100 kDa, at least 110 kDa, at least 120 kDa, at least 130 kDa, at least 140 kDa, at least 150 kDa, etc., up to about 10,000 kDa. However, the antibodies encapsulated and/or dispersed in the particles described herein typically have average molecular weights in the range of about 100 kDa or 150 kDa or 200 kDa up to about 1,500 kDa, or about 1.000 kDa.

i. Antibodies

The particles contain one or more antibodies dispersed and/or encapsulated therein. The term “antibody” is intended to denote an immunoglobulin molecule that possesses a variable region antigen recognition site. The term “variable region” is intended to distinguish such domain of the immunoglobulin from domains that are broadly shared by antibodies (such as an antibody Fc domain). The variable region includes a hypervariable region whose residues are responsible for antigen binding. The hypervariable region includes amino acid residues from a Complementarity Determining Region or CDR (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a hypervariable loop (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). Framework Region or FR residues are those variable domain residues other than the hypervariable region residues as herein defined.

The term “antibody” includes monoclonal antibodies, multispecific antibodies, human antibodies, humanized antibodies, synthetic antibodies, chimeric antibodies, camelized antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA, and IgY), class (e.g., IgG₁, IgG₂, IgG₃, IgG₄, IgA₁, and IgA₂) or subclass. Thus, the term “antibody” includes both intact molecules as well as fragments thereof that include the antigen-binding site and are capable of binding to the desired epitope. These include Fab and F(ab′)₂ fragments which lack the Fc fragment of an intact antibody, and therefore clear more rapidly from the circulation, and may have less non-specific tissue binding than an intact antibody (Wahl et al., J. Nuc. Med. 24:316-325 (1983)). Also included are Fv fragments (Hochman, J. et al., Biochemistry, 12:1130-1135 (1973); Sharon, J. et al., Biochemistry, 15:1591-1594 (1976)).

In some embodiments, the disclosed pharmaceutical compositions and methods are used to deliver a therapeutic antibody in a micronized form. Suitable therapeutic antibodies include, but are not limited to, those discussed in Reichert, Mabs, 3 (1): 76-99 (2011), for example, AIN-457, bapineuzumab, brentuximab vedotin, briakinumab, dalotuzumab, epratuzumab, farletuzumab, girentuximab (WX-G250), naptumomab estafenatox, necitumumab, obinutuzumab, otelixizumab, pagibaximab, pertuzumab, ramucirumab, REGN88, reslizumab, solanezumab, Tlh, teplizumab, trastuzumab emtansine, tremelimumab, vedolizumab (ENTYVIO®), zalutumumab and zanolimumab.

Other therapeutic antibodies approved for use in clinical trials or in development for clinical use that can be included in the particles for delivery include, but are not limited to, rituximab (Rituxan®, IDEC/Genentech/Roche) (see for example U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT/US2003/040426, entitled “Immunoglobulin Variants and Uses Thereof”), trastuzumab (Herceptin®, Genentech) (see for example U.S. Pat. No. 5,677,171), a humanized anti-Her2/neu antibody approved to treat breast cancer; pertuzumab (rhuMab-2C4, Omnitarge), currently being developed by Genentech; an anti-Her2 antibody described in U.S. Pat. No. 4,753,894: cetuximab (Erbitux®, Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252 (2): 549-60; Rodeck et al., 1987, J Cell Biochem. 35 (4): 315-20; Kettleborough et al., 1991, Protein Eng. 4 (7): 773-83); 1CR62 (Institute of Cancer Research) (PCT WO 95/20045; Modjtahedi et al., 1993, J. Cell Biophys. 1993, 22 (1-3): 129-46; Modjtahedi et al., 1993, Br J Cancer. 1993, 67 (2): 247-53; Modjtahedi et al, 1996, Br J Cancer, 73 (2): 228-35; Modjtahedi et al, 2003, Int J Cancer, 105 (2): 273-80); TheraCIM hR3 (YM Biosciences, Canada and Centro de Immunologia Molecular, Cuba (U.S. Pat. No. 5,891,996; U.S. U.S. Pat. No. 6,506,883; Mateo et al, 1997, Immunotechnology, 3 (1): 71-81); mAb-806 (Ludwig Institue for Cancer Research, Memorial Sloan-Kettering) (Jungbluth et al. 2003, Proc Natl Acad Sci USA. 100 (2): 639-44); KSB-102 (KS Biomedix); MRI-1 (IVAX, National Cancer Institute) (PCT WO 0162931A2); and SC100 (Scancell) (PCT WO 01/88138): alemtuzumab (Campath®. Millenium), a humanized mAb currently approved for treatment of B-cell chronic lymphocytic leukemia; muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amcvive®), anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFalpha antibody developed by Centocor, adalimumab (Humira®), an anti-TNFalpha antibody developed by Abbott, Humicade®, an anti-TNFalpha antibody developed by Celltech, golimumab (CNTO-148), a fully human TNF antibody developed by Centocor, etanercept (Enbrel®), an p75 TNF receptor Fc fusion developed by Immunex/Amgen, lenercept, an p55TNF receptor Fc fusion previously developed by Roche, ABX-CBL, an anti-CD 147 antibody being developed by Abgenix, ABX-IL8, an anti-IL8 antibody being developed by Abgenix, ABX-MAI, an anti-MUC18 antibody being developed by Abgenix, Pemtumomab (R1549,90Y-muHMFG1), an anti-MUCI in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegrene (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-.beta.2 antibody being developed by Cambridge Antibody Technology, ABT 874 (J695), an anti-IL-12 p40 antibody being developed by Abbott, CAT-192, an anti-TGF.beta.1 antibody being developed by Cambridge Antibody Technology and Genzyme, CAT-213, an anti-Eotaxinl antibody being developed by Cambridge Antibody Technology, LyntphoStat-B® an anti-Blys antibody being developed by Cambridge Antibody Technology and Human Genome Sciences Inc., TRAIL-RImAb, an anti-TRAIL-R1 antibody being developed by Cambridge Antibody Technology and Human Genome Sciences, Inc. Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech. Xolair® (Omalizurnab), an anti-IgE antibody being developed by Genentech, Raptiva® (Efalizurnab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDFC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (labetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-I), and anti-Her2 antibody being developed by Medarex and Immuno-Designed Molecules, HuMaxe-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J. CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAFO, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLN01, an anti-Beta2 integrin antibody being developed by Xoma. In another embodiment, the therapeutics include KRN330 (Kirin); huA 33 antibody (A33, Ludwig Institute for Cancer Research); CNTO 95 (alpha V integrins, Centocor); MEDI-522 (alpha V133 integrin, Medimmune); volociximab (aVB1 integrin, Biogen/PDL); Human mAb 216 (B cell glycosolated epitope, NCI); BiTE MT103 (bispecific CD19×CD3, Medimmune); 4G7×H22 (Bispecific BcellxFcgammaR1, Meclarex/Merck KGa); rM28 (Bispecific CD28×MAPG, EP1444268); MDX447 (EMD 82633) (Bispecific CD64×EGFR, Medarex); Catumaxomab (removah) (Bispecific EpCAM×anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase [X, Wilex); CNTO 888 (CCL2, Centocor); TRC105 (CD105 (endoglin), Tracon); BMS-663513 (CD137 agonist, Brystol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); THIOMAB (Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCI); MDX-060 (CD30. Medarex); MDX-1401 (CD30, Medarex); SGN-30 (CD30, Seattle Genentics); SGN-33 (Lintuzumab) (CD33, Seattle Genentics); Zanolimumab (HuMax-CD4) (CD4, Genmab); HCD 122 (CD40, Novartis); SGN-40 (CD40, Seattle Genentics); Campathlh (Alemtuzumab) (CD52, Genzyme); MDX-1411 (CD70, Medarex); hLL1 (EPB-I) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Brystol Myers Squibb): Tremelimumab (Ticilimumab, CP-675,2) (CTLA4, Pfizer); 1-IGS-ETRI (Mapatumumab) (DR4TRAIL-R1 agonist, Human Genome Science/Glaxo Smith Kline); AMG-655 (DR5, Amgen); Apomab (DR5, Genentech); CS-1008 (DR5, Daiichi Sankyo); HGS-ETR2 (lexatumumab) (DR5TRAIL-R2 agonist, HGS); Cetuximab (Erbitux) (EGFR, Imclone); IMC-11F8, (EGFR, Imclone); Nimotuzumab (EGFR, YM Bio); Panitumumab (Vectabix) (EGFR, Amgen); Zalutumumab (HuMaxEGFr) (EGFR, Genmab); CDX-110 (EGFRVIII, AVANT Immunotherapeutics); adecatumumab (MT201) (Epcam, Merck); edrecolomab (Panorex, 17-1A) (Epcam Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DR beta chain, PDL Pharma); AMG-479 (IGF-1R, Amgen); anti-IGF-1R R1507 (IGF1-R, Roche); CP 751871 (IGF 1-R, Pfizer); IMC-A12 (IGF1-R, Imclone); B1111022 Biogen); Mik-beta-1 (IL-2Rb (CD122), Hoffman LaRoche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTBR, Biogen); HuHMFG1 (MUCI, Antisoma/NCI); RAV 12 (N-linked carbohydrate epitope, Raven); CAL (parathyroid hormone-related protein (PTH-rP), University of California); CT-011 (PD1, CtireTech); MDX-1106 (ono-4538) (PDL Nileclarox/Ono); MAb CT-011 (PD1, Curetech); IMC-3G3 (PDGFRa, Imclone); bavituximab (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFα, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab-WO/2000/034337, University of Texas); IMC-18F1 (VEGFR1, Imclone); IMC-1121 (VEGFR2, Imclone).

a. Micronized Antibody

The antibody is typically micronized prior to incorporation into the microparticles or nanoparticles. “Micronization,” or other related terms, such as “micronized” or “micronizing,” generally refers to the production of particles in which the resulting particle size distribution is in the micrometer range, such as less than 10 μm. In some cases, the resulting particle size distribution is in the nanometer scale. The micronization can be performed by particle size reduction after the particle has been formed, or it can be in situ where micron-sized particles are obtained during generation of the particle without the need for further particle size reduction.

Following micronization, the micronized antibody contains less than 50% w/w, less than 40% w/w, less than 30% w/w, less than 20% w/w, less than 10% w/w, or less than 5% w/w excipients having a molecular weight of less than 20 kDa. Preferably, these excipients are non-polymeric. Typically, these excipients are used to stabilize an antibody in an aqueous buffer. These excipients are generally referred to herein as “antibody stabilizing excipient(s)”. In these forms, the antibody is dialyzed via 20 kDa molecular weight cut off dialysis cassette for at least 1 minute, such as for about 2 hours or about 24 hours, prior to or following micronization. Preferably, the dialyzation step occurs prior to the micronization process.

In some forms, the micronization can be via tert-butyl alcohol (TBA) micronization. In TBA micronization, the antibody is added to a solvent (e.g., water) to form a solution, dispersion, or suspension. The solution, dispersion, or suspension is added dropwise to pre-warmed TBA in a container. The volume ratios of the TBA with the solution, dispersion, or suspension can be adjusted to control the degree of micronization. In some forms, the ratio of the solution, dispersion, or suspension to TBA is about 1:10. Typically, 100 μL of the solution, dispersion, or suspension is added dropwise into 1 mL of TBA. The mixture of the solution, dispersion, or suspension and TBA is then bath sonicated for an appropriate time, such as about 1 minute. The mixture can be vortexed for an appropriate time, such as 40 seconds or until the mixture looks cloudy. The mixture can then be immediately placed in a cryogen, such as liquid nitrogen for flash freezing, and lyophilization, preferably overnight, to obtain the micronized antibody. The retrieved micronized antibody can be stored at −20° C. for later use. In some forms, the micronization steps are the same as above, except that instead of TBA solvent, a 0.1% (w/v) of a polymer (e.g., PLGA (6 kDa, 85:15 ratio)/PLA (2 kDa)) in dichloromethane is used. Other forms of micronization, such as cryo-emulsion, solvent evaporation, hot melt particle formation, solvent removal, spray drying, phase inversion, coacervation, low temperature casting, and film casting are known in the art, and can be readily implemented by those of skill in the art.

b. Antibody Size

Antibodies generally have an average molecular weight of between 100 kDa and 200 kDa, such as between 145 kDa and 160 kDa or between 150 kDa and 160 kDa. Generally, the micronized antibodies have an average molecular weight of between 100 kDa and 1500 kDa. Preferably, the micronized antibody has a molecular weight in the range of about 145 kDa and about 200 kDa, such as about 150 kDa.

c. Antibody Loading

The micronized antibody is typically dispersed and/or encapsulated in the microparticle or nanoparticle. The loading of the micronized antibody can be varied based on the specific antibody being delivered. The loading of the micronized antibody within the microparticle and/or nanoparticle is in a range from about 0.3% w/w to about 20% w/w, from about 0.3% w/w to about 15% w/w, or from about 0.3% w/w to about 10% w/w.

The micronized antibody in the pharmaceutical compositions may be a monodisperse or polydisperse population of particles. “Monodisperse” describes a population of nanoparticles or microparticles where all of the particles are the same or nearly the same size. As used herein, a monodisperse distribution refers to particle distributions in which at least 90% of the distribution lies within 5% of the median particle size. Polydisperse populations have greater variety in the size distribution of the particles compared to monodisperse populations. Preferably, the micronized antibody in the pharmaceutical compositions is monodispersed.

### 3. Controlled Release

The disclosed pharmaceutical compositions typically display controlled release properties. Upon reaching their target locale or systemically, the particles, preferably, release the agent to be delivered in a controlled release manner. The particles can provide sustained release of the active agent, for example for at least 24 hours, at least 36 hours, at least 48 hours, up to 100 hours, up to 200 hours, or up to 300 hours or longer following administration.

The types of polyhydroxyester polymers can be varied to tune the controlled release properties of the particles. For instance, PLA, PLGA, or their mixtures with different composition ratios can be used to control micronized antibody release. To this end, particles containing PLLA generally minimized or prevented zero-point burst release, regardless of their molecular weight. Further, particles containing PLLA generally also slowed down micronized antibody release, whereas particles containing PLGA appeared to increase release. Particles containing PLGA displayed faster, but prolonged release profiles. From these findings, combinations of PLLA and PLGA were tested. Particles containing PLLA showed none or the least burst release and even 10% w/w PLLA of 2 kDa in a particle suppressed the zero-point burst release. The ability to minimize or prevent zero-point burst release appeared to be in the order of PLLA 5 kDa, PLLA 2 kDa, PLGA 10 kDa (85_{L}: 15_{G}), and PLGA 4 kDa (50_{L}: 50_{G}).

### 4. Relative Bioavailability

Preferably, the disclosed pharmaceutical compositions typically increase the in vivo bioavailability or bioactivity of the antibody encapsulated and/or dispersed therein relative to the bioavailability of a control. Bioavailability can be determined by, for example, the serum level or serum half-life of the active agent following in vivo delivery. For example, an increase in the bioavailability can be demonstrated by an increase in serum concentration of the active agent over time or at a discrete time point relative to a control.

In some forms, the pharmaceutical compositions have improved bioactivity compared to a control. Bioactivity can be measured as, for example, a downstream pharmacological, physiological, or biochemical response to the active agent compared to a control.

The bioavailability and bioactivity of an antibody in the compositions can be compared to the same antibody delivered in the absence of particles, or via an alternative delivery system such as, for example, particles with the same or a similar size distribution that are formed from PLGA-COOH as the only polymer.

In some forms, the bioavailability of an active agent, such as a micronized antibody, can be determined by comparing the area under curve for isolated loop experiments (described in the Example section below) with the area under curve for a specified route of administration for the same time point (e.g., 0 hour to 5 hours) and dosage per animal (such as 0.75 mg of micronized antibody).

Briefly, blood is withdrawn from a rat's tail to establish baseline concentration (zero-point concentration). Then an incision is made on the ventral midline of the rat's abdomen and 10-20 cm of loop of a relevant gastrointestinal tract section is isolated and exteriorized. One side of the loop is ligated and the other side tied loosely. A solution, dispersion, or suspension containing a relevant pharmaceutical composition is injected into the loose end. Once the injection is complete, the loose end is tightened, thereby isolating the loop from the rest of the gastrointestinal tract. The isolated loop is then placed back into the abdominal cavity of the rat and the incision is closed. Blood samples can be collected via the rat's tail, for example, at the zero time point and 5 hours post-injection. The blood samples can be kept at room temperature and centrifuged for a given time at a suitable temperature (such as 4° C.) and under a suitable centrifugal force (such as 2000 g). Serum can be collected and analyzed or stored at an appropriate temperature before analysis. Another rat can also be administered the relevant pharmaceutical composition orally, intravenously, subcutaneously, or percutaneously, and blood samples collected at the zero time point and 5 hours post injection, the blood samples analyzed as in the rat in the isolated loop experiment, a plot of micronized antibody concentration in serum plotted at each time point, and the area under curve determined. The area under curve for this specified route is then compared with the area under curve in the isolated loop experiment to determine bioavailability.

In some forms, the bioavailability is compared to the same antibody not encapsulated nor dispersed in a particle, an in an aqueous solution containing a stabilizer for the antibody body. In some forms, the pharmaceutical composition at 5 hours following oral delivery is at least 40% of the bioavailability of a subcutaneously administered pharmaceutical composition containing the same antibody unencapsulated in an aqueous solution containing stabilizer for the antibody.

Additional details on how to determine bioavailability are provided in the Example section below.

### 5. Optional Features

i. Bioadhesivity

Optionally, the particles of the compositions are coated with polymers that are bioadhesive. A bioadhesive polymer is one that binds to mucosal epithelium under normal physiological conditions. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., van der Waals interactions) and stronger specific interactions, which include hydrogen bonds. The hydrophilic functional groups primarily responsible for forming hydrogen bonds are the hydroxyl and the carboxylic groups. Typically, adhesion occurs in an aqueous environment. Mucoadhesivity is a more specific form of bioadhesivity that refers to the interaction of a substance and the mucosal tissue. Bioadhesivity can be quantitated in relative terms, such as, but not limited to, a spectrum of bioadhesiveness within a group of substances, such as polymers and/or particle coated with such a polymer. In some forms where two or more polymers are being discussed, the terms “bioadhesivity” and “mucoadhesivity” can be defined based on a polymer's or particle's relative bioadhesiveness when compared to another, more bioadhesive polymer or particle, respectively. Bioadhesivity can be measured as described in Chickering and Mathiowitz, Journal of Controlled Release (1995), 34:251-261; U.S. Pat. No. 6,197,346 to Mathiowitz, et al.; and U.S. Pat. No. 6,235,313 to Mathiowitz, et al., the contents of which are hereby incorporated by reference.

Bioadhesives with varying hydration times and durations of bioadhesiveness in aqueous media could directly impact the performance of oral formulations. Bioadhesives have demonstrated the ability to promote intimate contact with the GI mucosa for prolonged periods of time leading to increased bioavailability of small molecule drugs. Additionally, it has been reported that a relationship exists between increased bioadhesiveness and increased particle uptake. Given the therapeutic aims of the oral formulation, taking into account the pharmacokinetics of the release and mucus turnover, choosing a polymer that will remain bioadhesive for the desired duration is of great importance to the field of oral drug delivery.

For example, to achieve prolonged release in the intestines of a small molecule over the period of hours, a bioadhesive with a low rate of hydration might be ideal, e.g. poly(fumaric-co-sebacic anhydride). However, as a carrier to enhance particle uptake, the bioadhesive polymer may function to promote contact between the particle and the GI mucosa for a short time until the particle can achieve mucus permeation and then dissolve prior to particle uptake.

Representative bioadhesive polymers include bioerodible hydrogels, such as those described by Sawhney, et al., Macromolecules, 1993, 26:581-587, and Estrellas, et al., Colloid. Surf. B Biointerfaces. 2019, 173, 454-469, the teachings of which are incorporated herein by reference. Other suitable bioadhesive polymers are described in U.S. Pat. No. 6,235,313 to Mathiowitz, et al., the teachings of which are incorporated herein by reference, and include poly(butadiene-maleic anhydride) (PBMA) polymer conjugated with phenylalanine (PBMAP), tyrosine (PBMAT), and DOPA (PBMAD); poly (ethylene-maleic anhydride) (PEMA) polymer conjugated with phenylalanine (PEMAP), tyrosine (PEMAT), and DOPA (PEMAD); polyhydroxy acids, such as PLA, PGA, and PLGA; polystyrene; polyhyaluronic acids; casein; gelatin; gluten; polyanhydrides; polyacrylic acid; alginate; chitosan; polyacrylates, such as poly(methyl methacrylates), poly(ethyl methacrylates), poly butylmethacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate); polyacrylamides; poly(fumaric-co-sebacic) acid; poly(bis carboxy phenoxy propane-co-sebacic anhydride); polyorthoesters; and combinations thereof.

Suitable polyanhydrides include polyadipic anhydride (“p(AA)”), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios.

In some forms, the bioadhesive material is a polymer containing a plurality of aromatic groups containing one or more hydroxyl groups. Such polymers are described in detail in U.S. Patent Publication No. 2005/0201974 to Schestopol, et al., the disclosure of which is incorporated herein by reference. Suitable aromatic moieties include, but are not limited to, catechol and derivatives thereof, trihydroxy aromatic compounds, or polyhydroxy aromatic moieties. Preferably, the aromatic moiety is catechol. In one embodiment, the aromatic moiety is 3,4-dihydroxyphenylalanine (L-DOPA), tyrosine, or phenylalanine, all of which contain a primary amine. In a particularly preferred embodiment, the aromatic compound is 3,4-dihydroxyphenylalanine.

The degree of substitution by the aromatic moiety can vary based on the desired adhesive strength; it may be as low as 10%, 20%, 25%, 50%, or up to 100% substitution. On average at least 50% of the monomers in the polymeric backbone are substituted with the at least one aromatic moiety. Preferably, 75-95% of the monomers in the backbone are substituted with at least one of the aromatic groups or a side chain containing one or more aromatic groups. In the preferred embodiment, on average 100% of the monomers in the polymeric backbone are substituted with at least one of the aromatic groups or a side chain containing one or more of the aromatic groups.

The bioadhesive polymer can be formed by first coupling the aromatic compound to a monomer or monomers and polymerizing the monomer or monomers to form the bioadhesive polymer. In this embodiment, the monomers may be polymerized to form any polymer, including biodegradable and non-biodegradable polymers. Alternatively, polymer backbones can be modified by covalently attaching the aromatic moieties to the polymer back bone. In those embodiments where the aromatic moieties are grafted to a polymer chain, the aromatic moieties can be part of a compound, side chain oligomer, and/or polymer.

Regardless of the mechanism, the monomer or polymer must contain one or more reactive functional groups which can react with the aromatic moiety to form a covalent bond. In one embodiment, the aromatic moiety contains an amino group and the monomer or polymer contains one or more amino reactive groups. Suitable amino reactive groups include, but are not limited to, aldehydes, ketones, carboxylic acid derivatives, cyclic anhydrides, alkyl halides, acyl azides, isocyanates, isothiocyanates, and succinimidyl esters.

The polymer that forms that backbone of the bioadhesive material containing the aromatic groups may be any non-biodegradable or biodegradable polymer. In the preferred embodiment, the polymer is a hydrophobic polymer. In one embodiment, the polymer is a biodegradable polymer.

Suitable polymer backbones include, but are not limited to, polyanhydrides, polyamides, polycarbonates, polyalkylenes, polyalkylene oxides such as polyethylene glycol, polyalkylene terepthalates such as poly(ethylene terephthalate), polyvinyl alcohols, polyvinyl ethers, polyvinyl esters, polyethylene, polypropylene, poly(vinyl acetate), poly vinyl chloride, polystyrene, polyvinyl halides, polyvinylpyrrolidone, polyhydroxy acids, polysiloxanes, polyurethanes and copolymers thereof, modified celluloses, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate).

ii. Carriers and Excipients

Optionally, the compositions may contain one or more pharmaceutically acceptable carriers or excipients in a media (solid or liquid) in which the nanoparticles and/or microparticles containing the antibodies are dispersed. Thus optionally, the antibody stabilizing excipients which were removed from the micronized antibodies, may be included in a medium in which these nanoparticles and/or microparticles are dispersed. In some forms, the pharmaceutically acceptable carriers or excipients can be included in the media to facilitate the formation of solid pharmaceutical formulations. The pharmaceutical formulations may be produced using standard procedures. Pharmaceutically carriers, excipients or diluents for different dosage forms are known in the art and described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). The disclosed compositions can be formulated with appropriate pharmaceutically acceptable carriers into pharmaceutical compositions for administration to an individual in need thereof. The compositions can be administered enterally (e.g., oral) or parenterally (e.g., by injection or infusion).

The disclosed compositions can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

In some embodiments, the pharmaceutical composition includes L-3,4-dihydroxyphenylalanine (L-DOPA). In some embodiments, when in a liquid carrier, the composition has an acidic pH, for example, about 1.5, about 2.5, about 3.5, about 4.5, about 5.5, about 6.5, up to about 6.9. Preferably, when in a liquid carrier, the composition has a pH between about 3.5 and about 6.9, more preferably about 4.0.

### 6. Exemplary Pharmaceutical Compositions

Exemplary pharmaceutical compositions with different release times are described in detail in the non-limiting examples and summarized herein.

In some forms of the pharmaceutical composition, the microparticle, nanoparticle, or a combination thereof contains between 1% w/w and 15% w/w micronized antibody and at least two of the polyhydroxyester polymers (i), (ii), and (iii):
(i) about 10% w/w to about 80% w/w PLLA of about 1 kDa to about 10 kDa or about 2 kDa to about 10 kDa;
(ii) about 10% w/w to about 80% w/w PDLA of about 1 kDa to about 10 kDa or about 2 kDa to about 10 kDa; and
(iii) about 10% w/w to about 80% w/w PLGA of about 1 kDa to about 120 kDa.

In some forms of the pharmaceutical composition, the microparticle, nanoparticle, or a combination thereof contains between 1% w/w and 15% w/w micronized antibody and at least two of the polyhydroxyester polymers (i), (ii), and (iii):
(i) about 10% w/w to about 80% w/w PLLA of about 1 kDa to about 10 kDa or about 2 kDa to about 10 kDa;
(ii) about 10% w/w to about 80% w/w PDLA of about 1 kDa to about 10 kDa or about 2 kDa to about 10 kDa; and
(iii) about 10% w/w to about 80% w/w PLGA of about 1 kDa to about 120 kDa,
wherein the percent weights of the polymers are the polymer weight ratios between the polyhydroxyester polymers. For example, in these forms, the weight ratio is polyhydroxyester (i):polyhydroxyester (ii); polyhydroxyester (i):polyhydroxyester (iii); polyhydroxyester (ii):polyhydroxyester (iii); or polyhydroxyester (i):polyhydroxyester (ii):polyhydroxyester (iii).

In some forms of the pharmaceutical composition, the microparticle, nanoparticle, or a combination thereof contains between 1% w/w and 15% w/w micronized antibody, and:
(i) about 35% w/w to about 80% w/w, about 50% w/w to about 80% w/w, or about 55 w/w to about 80% w/w PLLA of about 1 kDa to about 10 kDa or about 1 kDa to about 5 kDa, or about 2 kDa; and
(ii) about 20% w/w to 50% w/w, or about 20% w/w to about 45% w/w of about 1 kDa to about 120 kDa, about 4 kDa, about 10 kDa, about 38-54 kDa, or about 110 kDa PLGA, preferably wherein lactide and glycolide residues in the PLGA are present in a ratio of about 50:50 or about 85:15.

In some forms of the pharmaceutical composition, the microparticle, nanoparticle, or a combination thereof contains between 1% w/w and 15% w/w micronized antibody, and:
(i) about 35% w/w to about 80% w/w, about 50% w/w to about 80% w/w, or about 55 w/w to about 80% w/w PLLA of about 1 kDa to about 10 kDa or about 1 kDa to about 5 kDa, or about 2 kDa; and
(ii) about 20% w/w to 50% w/w, or about 20% w/w to about 45% w/w of about 1 kDa to about 120 kDa, about 4 kDa, about 10 kDa, about 38-54 kDa, or about 110 kDa PLGA, preferably wherein lactide and glycolide residues in the PLGA are present in a ratio of about 50:50 or about 85:15,
wherein the percent weights of the polymers are the polymer weight ratios between the polyhydroxyester polymers. For example, in these forms, the weight ratio is polyhydroxyester (i):polyhydroxyester (ii).

In some forms of the pharmaceutical composition, the microparticle, nanoparticle, or combination thereof contains at least two of the polyhydroxyester polymers (i), (ii), and (iii),
(i) 1-30% w/w PLLA of 2-10 kDa;
(ii) 1-50% w/w PDLA of 2-10 kDa;
(iii) 70-96% w PLGA of 2-60 kDa; and
(iv) 4-20% w/w micronized antibody loading. Preferably, in these forms, 80-100% of the micronized antibody is released following 24 hours in medium in vitro at a temperature of 37° C. and 1 atm pressure.

In some forms of the pharmaceutical composition, the microparticle, nanoparticle, or combination thereof contains at least two of the polyhydroxyester polymers (i), (ii), and (iii),
(i) 1-30% w/w PLLA of 2-10 kDa;
(ii) 1-50% w/w PDLA of 2-10 kDa;
(iii) 70-96% w PLGA of 2-60 kDa; and
(iv) 4-20% w/w micronized antibody loading, wherein the percent weights of the polymers are the polymer weight ratios between the polyhydroxyester polymers. For example, in these forms, the weight ratio is polyhydroxyester (i):polyhydroxyester (ii); polyhydroxyester (i):polyhydroxyester (iii); polyhydroxyester (ii):polyhydroxyester (iii); or polyhydroxyester (i):polyhydroxyester (ii):polyhydroxyester (iii). Preferably, in these forms, 80-100% of the micronized antibody is released following 24 hours in medium in vitro at a temperature of 37° C. and 1 atm pressure.

In some forms of the pharmaceutical composition, the microparticle, nanoparticle, or combination thereof contains at least two of the polyhydroxyester polymers (i), (ii), (iii), and (iv),
(i) 1-50% w/w PLLA of 2-10 kDa;
(ii) 1-50% w/w PDLA of 2-10 kDa
(iii) 1-70% w/w PLGA (85:15) of 2-60 kDa;
(iv) 0-30% w/w PLGA (50:50) of 2-60 kDa; and
(v) up to 15% w/w the micronized antibody loading. Preferably, in these forms, 40-100% of the micronized antibody is released by 200 hours in medium in vitro at a temperature of 37° C. and 1 atm pressure.

In some forms of the pharmaceutical composition, the microparticle, nanoparticle, or combination thereof contains at least two of the polyhydroxyester polymers (i), (ii), (iii), and (iv),
(i) 1-50% w/w PLLA of 2-10 kDa;
(ii) 1-50% w/w PDLA of 2-10 kDa
(iii) 1-70% w/w PLGA (85:15) of 2-60 kDa;
(iv) 0-30% w/w PLGA (50:50) of 2-60 kDa; and
(v) up to 15% w/w the micronized antibody loading, wherein the percent weights of the polymers are the polymer weight ratios between the polyhydroxyester polymers. For example, in these forms, the weight ratio is polyhydroxyester (i):polyhydroxyester (ii); polyhydroxyester (i):polyhydroxyester (iii); polyhydroxyester (i):polyhydroxyester (iv); polyhydroxyester (ii):polyhydroxyester (iii); polyhydroxyester (ii): polyhydroxyester (iv), polyhydroxyester (iii): polyhydroxyester (iv), polyhydroxyester (i): polyhydroxyester (ii):polyhydroxyester (iii), or combinations selected from polyhydroxyester (i), (ii), (iii) and (iv). Preferably, in these forms, 40-100% of the micronized antibody is released by 200 hours in medium in vitro at a temperature of 37° C. and 1 atm pressure.

In some forms, the pharmaceutical composition is in a form suitable for subcutaneous or intraperitoneal injection, and the microparticle, nanoparticle, or combination thereof contains:
(i) 50-100% w/w PLLA of 2-20 kDa;
(ii) 0-35% w/w PLGA (85:15) of 10-50 kDa; and
(iii) up to 15% w/w of the micronized antibody loading. Preferably, in these forms, 40-100% of the micronized antibody is released by one month in medium in vitro.

In some forms, the pharmaceutical composition is in a form suitable for subcutaneous or intraperitoneal injection, and the microparticle, nanoparticle, or combination thereof contains:
(i) 50-100% w/w PLLA of 2-20 kDa;
(ii) 0-35% w/w PLGA (85:15) of 10-50 kDa; and
(iii) up to 15% w/w of the micronized antibody loading, wherein the percent weights of the polymers are the polymer weight ratios between the polyhydroxyester polymers. For example, in these forms, the weight ratio is polyhydroxyester (i):polyhydroxyester (ii). Preferably, in these forms, 40-100% of the micronized antibody is released by one month in medium in vitro.

In some forms, the particles have a core-shell structure. Preferably, the core contains polyhydroxyester polymers, such as those described above under the section entitled Polymers or Exemplary Pharmaceutical Compositions. Examples of preferred biodegradable polymers for forming the shell include synthetic polymers such as poly hydroxy acids, such as polymers of lactic acid and glycolic acid, polyanhydrides, poly(ortho) esters, polyesters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In one embodiment, the shell polymer is a copolymer of maleic anhydride and butadiene containing DOPA, tyrosine, and/or phenyl alanine groups. In another embodiment, the polymer is a copolymer of maleic anhydride and ethylene containing DOPA, tyrosine, and/or phenyl alanine groups. Other suitable monomers that can be copolymerized with maleic anhydride include vinyl acetate and styrene.

The polymer may a bioadhesive polymer, such as one or more of those described above in the section entitled Bioadhesivity. The polymer may be a known bioadhesive polymer that is hydrophilic or hydrophobic. Hydrophilic polymers include CARBOPOL™ (a high molecular weight, crosslinked, acrylic acid-based polymers manufactured by NOVEON™), polycarbophil, cellulose esters, and dextran.

### III. Methods of Making Nanoparticles or Microparticles

Methods for forming microparticles or nanoparticles containing micronized antibodies are disclosed. The methods generally involve (i) forming a suspension or dispersion containing micronized antibodies, (ii) adding the suspension or dispersion to a solution containing a polyhydroxyester polymer to form a second suspension, dispersion, or solution, (iii) transferring this mixture to a non-solvent of the micronized antibody and/or polyhydroxyester polymer to form microparticles or nanoparticles, (iv) filtering the mixture, and/or (v) flash-freezing and/or lyophilizing the material retained in the filter to obtain to the microparticles or nanoparticles containing micronized antibody.

The antibody may be micronized prior to incorporation into the microparticles or nanoparticles. In some forms, the micronization is cryo-emulsion or TBA micronization. These micronization methods are described above.

Optionally, the microparticles or nanoparticles containing the antibody include one or more polymers, such as the biocompatible polymers described above. The identity and quantity of the one or more additional polymers can be selected, for example, to influence particle release kinetics, and particle stability, i.e., that time required for distribution to the site where delivery is desired, and the time desired for delivery. Pharmaceutically acceptable excipients, including pH modifying agents, disintegrants, preservatives, and antioxidants, can be incorporated in media (liquid or solid) in which the microparticles, nanoparticles, or combination thereof are dispersed.

In some forms, the microparticles, nanoparticles, or combination thereof, are made in a process that involves a mixture containing at least two of the polyhydroxyester polymers (i), (ii), and (iii):
(i) about 10% w/w to about 80% w/w PLLA of about 1 kDa to about 10 kDa or about 2 kDa to about 10 kDa;
(ii) about 10% w/w to about 80% w/w PDLA of about 1 kDa to about 10 kDa or about 2 kDa to about 10 kDa; and
(iii) about 10% w/w to about 80% w/w PLGA of about 1 kDa to about 120 kDa, wherein the percent weights of the polymers are the feed weight ratios between the polyhydroxyester polymers.

In some forms, the microparticle, nanoparticle, or combination thereof are made in a process that involves a mixture containing:
(i) about 35% w/w to about 80% w/w, about 50% w/w to about 80% w/w, or about 55 w/w to about 80% w/w PLLA of about 1 kDa to about 10 kDa or about 1 kDa to about 5 kDa, or about 2 kDa; and
(ii) about 20% w/w to 50% w/w, or about 20% w/w to about 45% w/w of about 1 kDa to about 120 kDa, about 4 kDa, about 10 kDa, about 38-54 kDa, or about 110 kDa PLGA, preferably wherein lactide and glycolide residues in the PLGA are present in a ratio of about 50:50 or about 85:15,
wherein the percent weights of the polymers are the feed weight ratios between the polyhydroxyester polymers.

In some forms, the microparticle, nanoparticle, or combination thereof, are made in a process that involves a mixture containing at least two of the polyhydroxyester polymers (i), (ii), and (iii):
(i) 1-30% w/w PLLA of 2-10 kDa;
(ii) 1-50% w/w PDLA of 2-10 kDa;
(iii) 70-96% w/w PLGA of 2-60 kDa,
wherein the percent weights of the polymers are the feed weight ratios between the polyhydroxyester polymers.

In some forms, the microparticle, nanoparticle, or combination thereof, are made in a process that involves a mixture containing at least two of the polyhydroxyester polymers (i), (ii), (iii), and (iv):
(i) 1-50% w/w PLLA of 2-10 kDa;
(ii) 1-50% w/w PDLA of 2-10 kDa
(iii) 1-70% w/w PLGA (85:15) of 2-60 kDa;
(iv) 0-30% w/w PLGA (50:50) of 2-60 kDa,
wherein the percent weights of the polymers are the feed weight ratios between the polyhydroxyester polymers.

In some forms, the microparticle, nanoparticle, or combination thereof, are made in a process that involves a mixture containing:
(i) 50-100% w/w PLLA of 2-20 kDa;
(ii) 0-35% w/w PLGA (85:15) of 10-50 kDa,
wherein the percent weights of the polymers are the feed weight ratios between the polyhydroxyester polymers.

The disclosed particles can be manufactured using any suitable known method in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). Preferably, the particles are made by phase inversion nanoencapsulation (“PIN”). U.S. Patent Application Publication US20040070093A1 by Mathiowitz, et al. and U.S. Pat. No. 6,123,965 to Jacob and Mathiowitz, describe phase inversion, the contents of which are hereby incorporated by reference.

Briefly, phase inversion is a physical process in which a polymer is first dissolved in “good” solvent, forming one continuous homogenous liquid phase. By adding this mixture to the excess of a non-solvent (or “bad” solvent), an unstable two-phase mixture of polymer rich and polymer poor fractions is formed, causing the polymer to aggregate at the nucleation points. When the polymer concentration reaches a certain point (cloud point), polymeric cores phase separate, solidifying and precipitating from the solution.

Unlike solvent removal or solvent evaporation methods, PIN does not require emulsification of the initial continuous phase polymer/solvent solution. It utilizes low polymer concentrations and low viscosities of the encapsulants. Also, the solvent and non-solvent pairs are preferably miscible with at least ten times excess of non-solvent relative to solvent. These conditions allow for rapid addition of polymer dissolved in continuous solvent phase into non-solvent, which in turn result in spontaneous formation of nanomaterial (e.g., nanoparticles) or micromaterial (e.g., microparticles). Since no emulsification is required in this process and the nanoparticles (e.g., nanospheres) or microparticles (e.g., microspheres) form spontaneously, the size of the resulting particles (e.g., size of the microspheres or nanospheres) is controlled not by the speed of stirring, but rather by changing the parameters of the procedure: polymer concentration, solvent to non-solvent ratio and their miscibility.

Briefly, to encapsulate and/or disperse micronized antibodies in microparticles or nanoparticles, a desired amount of polymer(s) is dissolved in a suitable organic solvent, such as dichloromethane (DCM) to make a solution. An alternative solvent can be chosen for this step, based on the solubility of the polymer(s) used. A known amount of as-prepared micronized antibody is added to the polymer solution to create suspension, dispersion, or another solution. The suspension, dispersion, or solution is then vortexed and bath sonicated. The resultant mixture can be kept at room temperature for a suitable time, such as at least 2 hours or overnight. Without wishing to be bound by theory, it is believed that the polymer chains will remain unentangled by this stabilization step. After brief vortexing and sonication, the micronized antibody-polymer mixture is added continuously by a glass pipette to an excess of an organic solvent (e.g., a non-solvent of the micronized antibody and/or polymer), such as petroleum ether (PE) with mild stirring. Preferably, the micronized antibody-polymer mixture to PE volume ratio is about 1:50 to 1:130, such as 1:50, 1:55, 1:60, 1:65, 1:70, 1:75, 1:80, or 1:125. If needed, the resultant mixture can be stirred for a suitable time, such as 5˜10 minutes with gentle stirring (100 rpm). The mixture can then be filtered through a filter such as a PTFE Millipore 0.2 μm filter by applying positive air pressure. The material retained in the filter can be left to dry under positive air pressure for an additional time, such as 5˜10 minutes. The filtered powder can be collected in a pre-weighted tube for flash freezing in a cryogen, such as liquid nitrogen, optionally followed by overnight lyophilization to remove residual solvent to obtain the micronized antibody encapsulated and/or dispersed in the microparticle or nanoparticle.

### IV. Methods of Using Pharmaceutical Compositions

The pharmaceutical compositions can be formulated into a variety of different micronized antibody delivery dosage forms and administered to a patient by any suitable method, including oral, injection (subcutaneous, percutaneous, intramuscular, intravenous, intraperitoneal), sublingual, inhalation, and transdermal administration. In particularly preferred methods, the pharmaceutical compositions are formulated for oral, subcutaneous, or percutaneous administration.

In some forms, the pharmaceutical compositions are formulated for systemic or local delivery via oral administration. In some forms, the pharmaceutical compositions are formulated for systemic or local delivery via subcutaneous administration. In some forms, the pharmaceutical compositions are formulated for systemic or local delivery via percutaneous administration. In the context of local delivery, the pharmaceutical compositions can be administered as a depot. For example, the depot could be injected or implanted orally, subcutaneously, or percutaneously to allow for controlled delivery of a micronized over a period, such as 24 hours, 48 hours, 72 hours, 100 hours, 200 hours, two weeks, one month, or longer.

The pharmaceutical compositions may be administered in an effective manner and amount to treat a variety of diseases, disorders and/or conditions, particularly those involving antibody-based therapies, i.e., treatments that use antibodies to assist the body to fight autoimmune disorders, cancers, infection, or other diseases. These diseases/disorders include, but are not limited to autoimmune disorders (e.g., Crohn's disease, ulcerative colitis, irritable bowel syndrome, celiac disease, rheumatoid arthritis, systemic lupus erythematosus, multiple sclerosis, type 1 diabetes mellitus, Guillain-Barre syndrome, psoriasis, etc.), and gastrointestinal cancers (e.g., gastric cancer, stromal tumors, lipomas hamartomas, and carcinoid syndromes).

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

The disclosed pharmaceutical compositions; methods of use; and methods of making microparticles, nanoparticles, or a combination thereof, can be further understood through the following numbered paragraphs.
1. A pharmaceutical composition for delivering an antibody containing a micronized antibody having a molecular weight of 100 kDa or greater encapsulated or dispersed in a microparticle, a nanoparticle, or a combination thereof containing one or more polyhydroxyester polymers having an average molecular weight of between about 2 kDa and about 30 kDa, inclusive, preferably wherein the composition provides sustained release of the micronized antibody with less than 10% of the micronized antibody released initially (0 hour).
2. The pharmaceutical composition of paragraph 1, wherein the microparticle has a size of between at least 1 μm and about 50 μm, between at least 1 μm and about 40 μm, between at least 1 μm and about 30 μm, between at least 1 μm and about 20 μm, between at least 1 μm and about 10 μm, between at least 1 μm and about 5 μm, or between at least 1 μm and about 2 μm.
3. The pharmaceutical composition of paragraph 1, wherein the nanoparticle has a size of between at least 50 nm and less than 1 μm, between at least 100 nm and less than 1 μm, between at least 200 nm and less than 1 μm, between at least 300 nm and less than 1 μm, between at least 400 nm and less than 1 μm, between at least 500 nm and less than 1 μm, or between at least 600 nm and less than 1 μm.
4. The pharmaceutical composition of any one of paragraphs 1 to 3, wherein the micronized antibody has a size less than 600 nm, less than 500 nm, between at least 20 nm and less than 600 nm, between at least 20 nm and less than 500 nm, between at least 20 nm and less than 400 nm, between at least 20 nm and less than 300 nm, between at least 20 nm and less than 200 nm, or between at least 20 nm and less than 100 nm.
5. The pharmaceutical composition of any one of paragraphs 1 to 4, wherein the micronized antibody has a loading in the microparticles and/or nanoparticles between 0.3% w/w and 20% w/w, optionally between 0.3% w/w and 15% w/w, optionally between 0.3% w/w and 10% w/w.
6. The pharmaceutical composition of any one of paragraphs 1 to 5, wherein the micronized antibody is released for at least 4 hours, at least 12 hours, at least 24 hours, at least 36 hours, at least 48 hours, up to 100 hours, up to 200 hours, or up to 300 hours or longer in vitro or following administration.
7. The pharmaceutical composition of any one of paragraphs 1-6 containing at least one polyhdroxyester polymer selected from poly-L-lactide (PLLA), poly-D-lactide (PDLA), poly-D,L-lactide, and poly(lactic-co-glycolic acid) (PLGA).
8. The pharmaceutical composition of paragraph 7, containing PLLA having an average molecular weight of about 10 kDa or less.
9. The pharmaceutical composition of paragraph 8, wherein the average molecular weight of the PLLA is about 2, about 5, or about 10 kDa.
10. The pharmaceutical composition of any one of paragraphs 1-9, containing PDLA having an average molecular weight in the range of about 2 to about 14 kDa.
11. The pharmaceutical composition of any one of paragraphs 7-10, containing poly(lactic acid-co-glycolic acid) (PLGA) having an average molecular weight range of about 1 kDa to about 120 kDa, about 2 kDa to about 110 kDa, about 2 to about 60 kDa, about 4 kDa to about 14 kDa, optionally about 6 to about 14 kDa.
12. The pharmaceutical composition of any one of paragraphs 7-11, containing PLGA having a lactic acid:glycolic acid ratio of 50:50 or 85:15.
13. The pharmaceutical composition of any one of paragraphs 1-12, wherein PLLA contains (i) at least 10% w/w of the polyhydroxyester polymer(s), or (ii) at least 10% w/w of the microparticle, nanoparticle, or combination thereof.
14. The pharmaceutical composition of any one of paragraphs 1-13, wherein the microparticle, nanoparticle, or combination thereof is formed of a single polymer.
15. The pharmaceutical composition of any one of paragraphs 1-14, wherein the micronized antibody contains less than 50% w/w, less than 40% w/w, less than 30% w/w, less than 20% w/w, less than 10% w/w, or less than 5% w/w antibody stabilizing excipients having a molecular weight of less than 20 kDa, preferably wherein the antibody stabilizing excipients are non-polymeric.
16. The pharmaceutical composition of any one of paragraphs 1-14, wherein the antibody is dialyzed via 20 kDa molecular weight cut off dialysis cassette for at least 1 min, such as for about 2 hours or about 24 hours, prior to or post-micronization.
17. The pharmaceutical composition of any one of paragraphs 1-13, 15, or 16, wherein the microparticle, nanoparticle, or combination thereof is formed of a blend comprising two or more polyhydroxyester polymers.
18. The pharmaceutical composition of paragraph 17, wherein the blend contains (i) a polyhydroxyester homopolymer and a polyhydroxyester co-polymer (such as PLGA, PDLLA, etc) or (ii) two or more polyhydroxyester homopolymers, such as a blend between PLLA and PLDA.
19. The pharmaceutical composition of paragraph 17 or 18, containing between 1% w/w and 15% w/w micronized antibody, and the microparticle, nanoparticle, or combination thereof contains at least two of the polyhydroxyester polymers (i), (ii), and (iii):
   (i) about 10% w/w to about 80% w/w PLLA of about 1 kDa to about 10 kDa or about 2 kDa to about 10 kDa;
   (ii) about 10% w/w to about 80% w/w PDLA of about 1 kDa to about 10 kDa or about 2 kDa to about 10 kDa; and
   (iii) about 10% w/w to about 80% w/w PLGA of about 1 kDa to about 120 kDa.
20. The pharmaceutical composition of paragraph 19, wherein the microparticle, nanoparticle, or combination thereof contains:
   (i) about 35% w/w to about 80% w/w, about 50% w/w to about 80% w/w, or about 55 w/w to about 80% w/w PLLA of about 1 kDa to about 10 kDa or about 1 kDa to about 5 kDa, or about 2 kDa; and
   (ii) about 20% w/w to 50% w/w, or about 20% w/w to about 45% w/w of about 1 kDa to about 120 kDa, about 4 kDa, about 10 kDa, about 38-54 kDa, or about 110 kDa PLGA, preferably wherein lactide and glycolide residues in the PLGA are present in a ratio of about 50:50 or about 85:15.
21. The pharmaceutical composition of paragraph 17, wherein the microparticle, nanoparticle, or combination thereof contains at least two of the polyhydroxyester polymers (i), (ii), and (iii),
   (i) 1-30% w/w PLLA of 2-10 kDa;
   (ii) 1-50% w/w PDLA of 2-10 kDa;
   (iii) 70-96% w PLGA of 2-60 kDa; and
   (iv) 4-20% w/w micronized antibody loading,
   preferably wherein 80-100% of the micronized antibody is released by 24 hours in medium in vitro.
22. The pharmaceutical composition paragraph 17, wherein the microparticle, nanoparticle, or combination thereof contains at least two of the polyhydroxyester polymers (i), (ii), (iii), and (iv),
   (i) 1-50% w/w PLLA of 2-10 kDa;
   (ii) 1-50% w/w PDLA of 2-10 kDa
   (iii) 1-70% w/w PLGA (85:15) of 2-60 kDa;
   (iv) 0-30% w/w PLGA (50:50) of 2-60 kDa; and
   (v) up to 15% w/w the micronized antibody loading,
   preferably wherein 40-100% of the micronized antibody is released by 200 hours in medium in vitro.
23. The pharmaceutical composition of paragraph 15 or 16 for subcutaneous or intraperitoneal injection, wherein the microparticle, nanoparticle, or combination thereof contains:
   (i) 50-100% w/w PLLA of 2-20 kDa;
   (ii) 0-35% w/w PLGA (85:15) of 10-50 kDa; and
   (iii) up to 15% w/w of the micronized antibody loading,
   preferably, wherein 40-100% of the micronized antibody is released by one month in medium in vitro.
24. The pharmaceutical composition of any of paragraphs 1-23, wherein the bioavailability of the formulation at 5 hours following oral delivery is at least 40% of the bioavailability of a subcutaneously administered formulation containing the same antibody unencapsulated in an aqueous solution containing stabilizer for the antibody.
25. The pharmaceutical composition of any one of paragraphs 1-23, wherein the particle, nanoparticle, or combination thereof further contains a bioadhesive coating on the surface of the microparticle and/or nanoparticle.
26. The pharmaceutical composition of paragraph 25, wherein the bioadhesive coating contains catechol functionalities.
27. The pharmaceutical composition of paragraph 26, wherein the catechol functionality is L-DOPA.
28. The pharmaceutical composition of any one of paragraphs 1-27, wherein (i) the microparticle has a size ranging from about 1 μm to about 50 μm, or (ii) the nanoparticle has a size ranging from about 300 nm to less than 1 μm.
29. The pharmaceutical composition of paragraph 28, wherein the microparticle has a size ranging from about 2 μm to about 10 μm.
30. The pharmaceutical composition of paragraph 28, wherein (i) the microparticle has a size ranging from about 1 μm to about 2 μm, or (ii) the nanoparticle has a size ranging from about 300 nm to less than 1 μm.
31. The pharmaceutical composition of any one of paragraphs 1 to 20, wherein the microparticle, nanoparticle, or combination thereof, is formed from a mixture contains at least two of the polyhydroxyester polymers (i), (ii), and (iii) having the following polymer weight ratios between the polyhydroxyester polymers:
   (i) about 10% w/w to about 80% w/w PLLA of about 1 kDa to about 10 kDa or about 2 kDa to about 10 kDa;
   (ii) about 10% w/w to about 80% w/w PDLA of about 1 kDa to about 10 kDa or about 2 kDa to about 10 kDa; and
   (iii) about 10% w/w to about 80% w/w PLGA of about 1 kDa to about 120 kDa.
32. The pharmaceutical composition of any one of paragraphs 1 to 17, wherein the microparticle, nanoparticle, or combination thereof contains 4-20% w/w micronized antibody loading, and the microparticle, nanoparticle, or combination thereof is formed from a mixture containing at least two of the polyhydroxyester polymers (i), (ii), and (iii) having the following polymer weight ratios between the polyhydroxyester polymers:
   (i) 1-30% w/w PLLA of 2-10 kDa;
   (ii) 1-50% w/w PDLA of 2-10 kDa; and
   (iii) 70-96% w PLGA of 2-60 kDa.
33. The pharmaceutical composition of any one of paragraphs 1 to 17, wherein the microparticle, nanoparticle, or combination thereof contains up to 15% w/w micronized antibody loading, and the microparticle, nanoparticle, or combination thereof is formed from a mixture containing at least two of the polyhydroxyester polymers (i), (ii), (iii), and (iv) having the following polymer weight ratios between the polyhydroxyester polymers:
   (i) 1-50% w/w PLLA of 2-10 kDa;
   (ii) 1-50% w/w PDLA of 2-10 kDa;
   (iii) 1-70% w/w PLGA (85:15) of 2-60 kDa; and
   (iv) 0-30% w/w PLGA (50:50) of 2-60 kDa.
34. The pharmaceutical composition of paragraph 15 or 16 for subcutaneous or intraperitoneal injection, wherein the microparticle, nanoparticle, or combination thereof contains up to 15% w/w micronized antibody loading, and the microparticle, nanoparticle, or combination thereof is formed from the polyhydroxyester polymers (i) and (ii) having the following polymer weight ratios between the polyhydroxyester polymers:
   (i) 50-100% w/w PLLA of 2-20 kDa; and
   (ii) 0-35% w/w PLGA (85:15) of 10-50 kDa.
35. The pharmaceutical composition of any one of paragraphs 1-34, wherein the microparticle, nanoparticle, or combination thereof, is formed by phase inversion nanoencapsulation (PIN).
36. The pharmaceutical composition of any one of paragraphs 1-35, wherein the microparticle, nanoparticle, or combination thereof is in a carrier containing a pharmaceutically acceptable carrier or excipient for oral or parenteral administration, optionally wherein the parenteral administration is subcutaneous, intravenous, or intraperitoneal.
37. The pharmaceutical composition of paragraph 36, wherein the carrier or excipient is L-DOPA.
38. The pharmaceutical composition of any one of paragraphs 1-37, in a liquid carrier, wherein the composition has an acidic pH.
39. The pharmaceutical composition of paragraph 38, wherein the pH is between about 3.5 and 6.9, preferably about 4.0.
40. The pharmaceutical composition of any one of paragraphs 1-39, wherein the antibody has a molecular weight of at least about 150 kDa.
41. The pharmaceutical composition of any of paragraphs 1-40, in a form suitable for systemic delivery via oral administration.
42. The pharmaceutical composition of any of paragraphs 1-38, in a form suitable for local delivery to the gastrointestinal tract via oral administration.
43. The pharmaceutical composition of any of paragraphs 1-40, in a form suitable for systemic delivery via subcutaneous administration.
44. The pharmaceutical composition of any of paragraphs 1-40, in a form suitable for systemic delivery via percutaneous administration to the gastrointestinal tract.
45. The pharmaceutical composition of any of paragraphs 1-40, in a form suitable for local delivery to the gastrointestinal tract via percutaneous administration.
46. A method of treating a subject in need thereof comprising administering to the subject the pharmaceutical composition of any one of paragraphs 1-45.
47. The method of paragraphs 46, wherein treating the subject involves antibody-based therapy.
48. A method of making a microparticle, a nanoparticle, or a combination thereof, containing a micronized antibody having a molecular weight of 100 kDa or greater encapsulated or dispersed in the microparticle, the nanoparticle, or combination thereof, the method involving mixing the micronized antibody in a solvent containing at least two of the polyhydroxyester polymers (i), (ii), and (iii):
   (i) about 10% w/w to about 80% w/w PLLA of about 1 kDa to about 10 kDa or about 2 kDa to about 10 kDa;
   (ii) about 10% w/w to about 80% w/w PDLA of about 1 kDa to about 10 kDa or about 2 kDa to about 10 kDa; and
   (iii) about 10% w/w to about 80% w/w PLGA of about 1 kDa to about 120 kDa, to form a solution, suspension, or dispersion, wherein the percent weights of the polymers are the feed weight ratios between the polyhydroxyester polymers.
49. A method of making a microparticle, a nanoparticle, or a combination thereof, containing a micronized antibody having a molecular weight of 100 kDa or greater encapsulated or dispersed in the microparticle, the nanoparticle, or combination thereof, the method involving
   mixing the micronized antibody in a solvent comprising at least two of the polyhydroxyester polymers (i), (ii), and (iii):
   (i) 1-30% w/w PLLA of 2-10 kDa;
   (ii) 1-50% w/w PDLA of 2-10 kDa;
   (iii) 70-96% w/w PLGA of 2-60 kDa, to form a solution, suspension, or dispersion, wherein the percent weights of the polymers are the feed weight ratios between the polyhydroxyester polymers.
50. A method of making a microparticle, a nanoparticle, or a combination thereof, containing a micronized antibody having a molecular weight of 100 kDa or greater encapsulated or dispersed in the microparticle, the nanoparticle, or combination thereof, the method involving
   mixing the micronized antibody in a solvent comprising at least two of the polyhydroxyester polymers (i), (ii), (iii), and (iv):
   (i) 1-50% w/w PLLA of 2-10 kDa;
   (ii) 1-50% w/w PDLA of 2-10 kDa;
   (iii) 1-70% w/w PLGA (85:15) of 2-60 kDa;
   (iv) 0-30% w/w PLGA (50:50) of 2-60 kDa, to form a solution, suspension, or dispersion, wherein the percent weights of the polymers are the feed weight ratios between the polyhydroxyester polymers.
51. A method of making a microparticle, a nanoparticle, or a combination thereof, containing a micronized antibody having a molecular weight of 100 kDa or greater encapsulated or dispersed in the microparticle, the nanoparticle, or combination thereof, the method involving
   mixing the micronized antibody in a solvent comprising the polyhydroxyester polymers:
   (i) 50-100% w/w PLLA of 2-20 kDa;
   (ii) 0-35% w/w PLGA (85:15) of 10-50 kDa, to form a solution, suspension, or dispersion, wherein the percent weights of the polymers are the feed weight ratios between the polyhydroxyester polymers.

### EXAMPLES

### Example 1: In Vitro Release Experiments with Non-Dialyzed and Dialyzed Antibody

### 1.1 Materials and Methods

### 1.1.1 Dialysis of Initial Drug Substance

The initial drug substance, Entyvio (vedolizumab) antibody was provided by Takeda and each vial contained ˜89 to 95 mg/mL for a total of ˜331.2 mg MLN002 antibody (71.7% wt human antibody), 16.002 mg Histidine (3.5% wt), 14.921 mg Histidine HCl (3.2% wt), 91.795 mg Arginine HCl (21.2% wt) and 2.092 mg Polysorbate 80 (0.5% wt). Prior to micronization, the initial drug substance was diluted to a concentration of 1 mg/mL of antibody in Takeda buffer (TB). Takeda buffer (TB) contained 50 mM Histidine, 125 mM arginine and 0.06% Polysorbate 80 adjusted to pH 6.3.

Formulations F1-F51 were micronized without a dialysis step prior to micronization (referred to herein as “non-dialyzed formulations”).

Formulations F52-F83 were prepared using an initial dialysis step prior to micronization (referred to herein “dialyzed formulations”). For the dialysis step, the initial drug substance was dialyzed for 24 hours using a 20 kDa MWCO dialysis cassette, then extracted and re-dissolved in milliQ water. Following dialysis, the concentration of the dialyzed drug was measured using High Performance Liquid Chromatography (HPLC) by dissolving 1 mg of the dried dialyzed drug in 1 mL Takeda buffer and injecting the solution into the HPLC column. The drug concentration was determined by comparing the peak of the dialyzed drug curve to a calibration curve generated using various concentrations of the initial drug substance. Following dialysis, the drug substance contained at least 50% (wt) of antibody; some loss of antibody was expected from the dialysis step. The peaks of the dialyzed drug were considered to reflect the presence of monomers, although dimers and/or trimers may be present, albeit in negligible amounts. Therefore, the actual dialyzed drug concentration may be higher than 50-60%.

### 1.1.2 Micronization of the Antibody

The starting material for the drug substance was in the form of a solution and was lyophilized. A drug solution was made by diluting the resulting drug substance with milliQ water to concentrations of either 1 mg/mL or 40-50 mg/mL. The drug solution was then added to approximately 10 mL of warm terbutylalcohol (TBA) and vortexed for 40 seconds. The drug solution was sonicated in a bath sonicator for 20 seconds after which it is vortexed again for 30 seconds then immediately flash frozen in liquid nitrogen and lyophilized.

The resulting micronized antibody was then encapsulated using the phase inversion nanoencapsulation (PIN) method.

### 1.1.3 Phase Inversion Nanoencapsulation (PIN)

Following micronization, the micronized drug was encapsulated at different drug loadings in different polymers or combinations of polymers via Phase Inversion Nanoencapsulation (PIN) to produce different formulations. The polymers and drug loadings for each formulation are listed in Tables 1 and 2.

First, 200 mg of the dry micronized drug and the desired amount of the polymer(s) were weighed in a 20 mL glass scintillation vial and dichloromethane (DCM) was added to yield a polymer solution of ˜1.5% w/v (for the formulations denoted with “F”) ˜2.5% w/v (for the formulations denoted with “FD”). The amount of drug used for encapsulation depends on the desired loading amount. However, efficiency of the encapsulation process declines with increasing drug loading amounts. At this stage, the polymer was soluble while the drug was insoluble, resulting in a murky polymer solution. The mixture of micronized drug in the polymer solution was vortexed for 40 seconds and sonicated for 20 seconds. The resulting polymer and micronized drug solution was then left for a minimum of 3 hours, preferably overnight. Next, the polymer and the micronized drug solution was briefly vortexed and sonicated, and then added dropwise to an excess of petroleum ether (PE) at a volume ratio of at least 1:60 polymer and micronized drug solution to PE. The addition of the polymer and the micronized drug solution to the PE solution was conducted using a glass pipette dipped in the PE solution while mixing gently with a magnetic stir bar. The solutions were mixed using the magnetic stir bar at 100 RPM for about 5 to 8 minutes.

The resulting encapsulated nanoparticles (NPs) were harvested onto a PTFE Millipore 0.22 μm filter using positive air pressure. The filter was dried under positive air pressure for an additional 5 to 7 minutes and the resulting encapsulated NPs were collected with a blank paper into a pre-weighted falcon tube. The falcon tube with the encapsulated NPs was flash frozen in liquid nitrogen and dried in the lyophilizer overnight to remove solvent residues. After lyophilization, the falcon tube was weighed to determine the yield. The final product was in the form of a powder and was kept in a −20° C. in a parafilm sealed vial.

TABLE 1
Polymers and Drug Loading for Microparticle formulations F1-F41 and F43-F46

| | Polymer | | | | | PLGA₁ | | | PLGA₂ | Drug |
|---|---|---|---|---|---|---|---|---|---|---|
| Formulation | Ratio | PLLA₁ | | PLLA₂ | D, L PLA | 50:50 | | PLLA₃ | 85:15 | Loading |
| No. | (% Mass) | (MW) | | (MW) | (MW) | (MW) | | (MW) | (MW | (wt %) |
| FD1 | 75:25 | 2 | kDa | — | — | 4 | kDa | — | — | ~2% |
| F2:1 | 67:33 | 2 | kDa | — | — | 4 | kDa | — | — | ~2.6% |
| FD2 | 60:40 | 2 | kDa | — | — | 4 | kDa | — | — | ~2% |

| | | | | | | | | | | |
|---|---|---|---|---|---|---|---|---|---|---|
| FD3 | 75:25 | — | — | 18 | kDa | 4 | kDa | — | — | ~2% |
| FD4 | 60:40 | — | | 18 | kDa | 4 | kDa | — | — | ~2% |

| | | | | | | | | | | |
|---|---|---|---|---|---|---|---|---|---|---|
| FD5 | 75:25 | 2 | kDa | — | — | 38 | kDa | — | — | ~2% |
| FD6 | 60:40 | 2 | kDa | — | — | 38 | kDa | — | — | ~2% |

| | | | | | | | | | | |
|---|---|---|---|---|---|---|---|---|---|---|
| FD7 | 75:25 | — | — | 18 | kDa | 38 | kDa | — | — | ~2% |
| FD8 | 60:40 | — | — | 18 | kDa | 38 | kDa | — | — | ~2% |

| | | | | | | | | | | |
|---|---|---|---|---|---|---|---|---|---|---|
| FD9 | 75:25 | 2 | kDa | — | — | 110 | kDa | — | — | ~2% |
| FD10 | 60:40 | 2 | kDa | — | — | 110 | kDa | — | — | ~2% |

| | | | | | | | | | | |
|---|---|---|---|---|---|---|---|---|---|---|
| FD11 | 75:25 | — | — | 18 | kDa | 110 | kDa | — | — | ~2% |
| FD12 | 60:40 | — | — | 18 | kDa | 110 | kDa | — | — | ~2% |
| FD13 | 70:30 | — | — | 8 | kDa | 4 | kDa | — | — | ~2% |
| FD14 | 70:30 | — | — | 8 | kDa | 38 | kDa | — | — | ~2% |
| FD15 | 70:30 | — | — | 8 | kDa | 110 | kDa | — | — | ~2% |

| | | | | | | | | | | |
|---|---|---|---|---|---|---|---|---|---|---|
| FD16 | 75:25 | 10 | kDa | — | — | — | — | 10 | kDa | ~2% |
| FD17 | 60:40 | 10 | kDa | — | — | — | — | 10 | kDa | ~2% |

| | | | | | | | | | | |
|---|---|---|---|---|---|---|---|---|---|---|
| FD18 | 75:25 | 10 | kDa | — | — | 38 | kDa | — | — | ~2% |
| F19 | 50:50 | 2 | kDa | — | — | 4 | kDa | — | — | ~2.9% |
| FD19 | 60:40 | 10 | kDa | — | — | 38 | kDa | — | — | ~2% |
| F20 | 75:25 | 2 | kDa | — | — | 4 | kDa | — | — | ~2.6% |
| F24 | 75:25 | 2 | kDa | — | — | 38 | kDa | — | — | ~2% |

| | | | | | | | | | | |
|---|---|---|---|---|---|---|---|---|---|---|
| F25 | 75:25 | 2 | kDa | — | — | — | — | 10 | kDa | ~2% |

| F26 | — | 2 | kDa | — | — | — | | — | — | ~4.1% |
|---|---|---|---|---|---|---|---|---|---|---|
| F27 | 60:40 | 10 | kDa | — | — | 4 | kDa | — | — | ~2% |

| | | | | | | | | | | | |
|---|---|---|---|---|---|---|---|---|---|---|---|
| F28 | 50:25:25 | 10 | kDa | 2 | kDa | — | — | — | 10 | kDa | ~2% |

| | | | | | | | | | | |
|---|---|---|---|---|---|---|---|---|---|---|
| F29 | 60:40 | 10 | kDa | — | — | 4 | kDa | — | — | ~3% |

| | | | | | | | | | | |
|---|---|---|---|---|---|---|---|---|---|---|
| F30 | 50:50 | 10 | kDa | 2 | kDa | — | — | — | — | ~2% |

| | | | | | | | | | | | |
|---|---|---|---|---|---|---|---|---|---|---|---|
| F31 | 60:20:20 | 2 | kDa | — | — | 110 | kDa | — | 4 | kDa | ~2% |

| | | | | | | | | | | |
|---|---|---|---|---|---|---|---|---|---|---|
| F32 | 75:25 | 2 | kDa | — | — | 38 | kDa | — | — | 4.1% GCA |
| F33 | 75:25 | 2 | kDa | — | — | 38 | kDa | — | — | 1% GCA |
| F34 | 60:40 | 2 | kDa | — | — | 4 | kDa | — | — | ~3% |
| F35 | 30:70 | 10 | kDa | — | — | 4 | kDa | — | — | ~2% |

| | | | | | | | | | | |
|---|---|---|---|---|---|---|---|---|---|---|
| F36 | 30:70 | 10 | kDa | — | — | — | — | 10 | kDa | ~2% |

| F37 | 30:70 | 10 | kDa | — | | — | 14 | kDa | — | — | ~2% |
|---|---|---|---|---|---|---|---|---|---|---|---|
| F38 | 10:45:45 | 10 | kDa | 2 | kDa | — | 4 | kDa | — | — | ~2% |
| F39 | 20:40:40 | 10 | kDa | 2 | kDa | — | 4 | kDa | — | — | ~2% |
| F40 | 40:30:30 | 10 | kDa | 2 | kDa | — | 4 | kDa | — | — | ~2% |

| | | | | | | | | | | | |
|---|---|---|---|---|---|---|---|---|---|---|---|
| F41 | 15:70:15 | 10 | kDa | — | — | 38 | kDa | 2 | kDa | — | ~2% |
| F43 | 15:70:15 | 10 | kDa | — | — | 14 | kDa | 2 | kDa | — | ~2% |

| | | | | | | | | | |
|---|---|---|---|---|---|---|---|---|---|
| F44 | 15:70:15 | 10 | kDa | 8 | kDa | 2 | kDa | — | ~2% |
| F45 | 15:70:15 | 10 | kDa | 18 | kDa | 2 | kDa | — | ~2% |

| | | | | | | | | | |
|---|---|---|---|---|---|---|---|---|---|
| F46 | 15:70:15 | 10 | kDa | 18 | kDa | 2 | kDa | — | ~2% |

In addition to the formulations described in Table 1 above, nine more formulations were produced and tested. Formulation F42 was generated using PLLA (10 kDa), PLGA 85:15 (10 kDa) and PLLA (2 kDa) in a weight ratio of 15:70:15 (i.e. 15% mass PLLA (10 kDa) to 70% mass PLGA 85:15 (10 kDa) to 15% mass PLLA (2 kDa)). Formulation F47 contained PLLA (10 kDa), PLGA 65:35 (10 kDa) and PLLA (2 kDa) at a weight ratio of 15:70:15. Formulation F48 was produced using PLLA (10 kDa), PLGA 85:15 (10 kDa) and PLLA (2 kDa) at a weight ratio of 20:60:20. Formulation F49 contained PLLA (10 kDa), PLGA 75:25 (8 kDa ester end) and PLLA (2 kDa) at a weight ratio of 15:60:15. Formulation F50 was created using PLLA (10 kDa), PLGA 75:25 (8 kDa) and PLLA (2 kDa) at a weight ratio of 15:70:15. Formulation F51 was generated using PLLA (10 kDa), PLGA 85:15 (6 kDa) and PLLA (2 kDa) at a weight ratio of 15:70:15.

TABLE 2
Polymers and Drug Loading for Microparticle formulations F52-F81

| | | | | PLGA₁ | | PLGA₂ | | | PLGA₃ | Drug |
|---|---|---|---|---|---|---|---|---|---|---|
| Formulation | Polymer Ratio | PLLA₁ | | 85:15 | | 50:50 | PLLA₂ | | 50:50 | Loading |
| No. | (% Mass) | (MW) | | (MW) | | (MW) | (MW) | | (MW | (wt %) |
| F52 | 60:40 | 2 | kDa | 10 | kDa | — | — | | — | 0.3% |
| F53 | 30:40:30 | 10 | kDa | 10 | kDa | — | 2 | kDa | — | 0.3% |

| | | | | | | | | | | | |
|---|---|---|---|---|---|---|---|---|---|---|---|
| F54 | 25:35:25:15 | 10 | kDa | — | 14 | kDa | 2 | kDa | 4 | kDa | 0.3% |

| F52H | 60:40 | 2 | kDa | 10 | kDa | — | — | | — | 2% |
|---|---|---|---|---|---|---|---|---|---|---|
| F53H | 30:40:30 | 10 | kDa | 10 | kDa | — | 2 | kDa | — | 2% |

| | | | | | | | | | | | |
|---|---|---|---|---|---|---|---|---|---|---|---|
| F54H | 25:35:25:15 | 10 | kDa | — | 14 | kDa | 2 | kDa | 4 | kDa | 2% |

| F55 | 65:35 | 5 | kDa | — | | 4 | kDa | — | — | ~2.0% |
|---|---|---|---|---|---|---|---|---|---|---|
| F56 | 65:35 | 5 | kDa | 10 | kDa | — | | — | — | ~2.0% |
| F57 | 50:50 | 2 | kDa | 10 | kDa | — | | — | — | 3.95% |
| F58 | 50:20:30 | 2 | kDa | 10 | kDa | 4 | kDa | — | — | 2.6% |
| F59 | 50:50 | 2 | kDa | — | | 4 | kDa | — | — | 2.7% |
| F60 | — | 2 | kDa | — | | — | | — | — | 4.2% |
| F61 | — | 2 | kDa | — | | — | | — | — | 14.8% |
| F62 | 25:75 | 2 | kDa | — | | 4 | kDa | — | — | ~2.0% |
| F63 | 25:75 | 2 | kDa | 10 | kDa | — | | — | — | ~2.0% |
| F64 | 25:60:15 | 2 | kDa | 10 | kDa | 4 | kDa | — | — | ~2.0% |
| F65 | 25:50:25 | 2 | kDa | 10 | kDa | 4 | kDa | — | — | ~2.0% |
| F66 | 25:40:35 | 2 | kDa | 10 | kDa | 4 | kDa | — | — | ~2.0% |
| F67 | 25:30:45 | 2 | kDa | 10 | kDa | 4 | kDa | — | — | ~2.0% |
| F68 | 60:40 | — | | 10 | kDa | 4 | kDa | — | — | ~2.0% |
| F69 | — | | | 10 | kDa | — | | — | — | ~2.0% |
| F70 | 15:60:25 | 2 | kDa | 10 | kDa | 4 | kDa | — | — | ~2.0% |
| F71 | 15:55:30 | 2 | kDa | 10 | kDa | 4 | kDa | — | — | ~2.0% |
| F72 | 10:65:25 | 2 | kDa | 10 | kDa | 4 | kDa | — | — | ~2.0% |
| F73 | 10:65:25 | 5 | kDa | 10 | kDa | 4 | kDa | — | — | ~2.0% |

| F74 | 45:55 | 2 | kDa | 10 | kDa | — | — | — | ~2.0% |
|---|---|---|---|---|---|---|---|---|---|
| F75 | — | 5 | kDa | — | | — | — | — | ~2.0% |
| F76 | 50:50 | 2 | kDa | 6 | kDa | — | — | — | ~2.0% |
| F77 | — | 2 | kDa | — | | — | — | — | 1.9% |
| F78 | — | — | | 6 | kDa | — | — | — | ~2.0% |
| F79 | 70:30 | 2 | kDa | 6 | kDa | — | — | — | ~2.0% |
| F80 | 50:50 | 2 | kDa | 6 | kDa | — | — | — | ~2.0% |

| | | | | | | | | |
|---|---|---|---|---|---|---|---|---|
| F81 | — | 2 | kDa | — | — | — | — | 7.8% |

In addition to the formulations described in Table 2 above, two additional formulations were produced and tested. Formulation F82 was generated using D, L PLA (8 kDa) with a drug loading of 0.9% (wt). Formulation F83 was composed of two PLA polymers at a ratio of 75:25 with 75% mass D, L PLA (8 kDa) and 25% mass PLLA (2 kDa) and a drug loading of 1.0% (wt).

### 1.1.4 In Vitro Release of Encapsulated Antibody Over Time

The in vitro release profile of the encapsulated antibody in formulations F1-F83, described in table. First 16-20 mg of the resulting nanoparticle powder for each of the formulations was placed in a 1.5 mL Eppendorf tube and 1.3 mL of Takeda buffer was added. The mixture of each powdered formulation and Takeda buffer was vortexed for 20 seconds and sonicated for 5-10 seconds for a total of at least three cycles until the nanoparticles were homogenously dispersed. More than three vortex and sonication cycles were used when most of the particles were not resuspended in the solution. Sampling was conducted by centrifuging the drug powder and Takeda buffer mixture at 10000 g for 8 minutes at 4° C. and removing 0.9 mL of the supernatant without disturbing the pellet. The drug concentration at each time point for the solution was measured in triplicate using HPLC analysis. The pellet was resuspended by adding 0.9 mL Takeda buffer then vortexed for 20 seconds and, if necessary, briefly sonicated.

Sampling of the zero time point was taken immediately after resuspending the particles. Additional samples were taken at 2, 3, 4, 5, 6, and 24 hours and in some experiments, daily for one week followed by once weekly thereafter. Throughout the experiment, the vials were kept on a rotator at room temperature (20-25° C.).

### 1.1.5 HPLC Measurements of Drug Concentration in Solutions

HPLC was used to obtain a reliable detection of Entyvio (vedolizumab or MLN0002) in solutions using an isocratic elution in phosphate-buffered saline (PBS) to separate aggregates, intact protein, and low MW species. MLN0002 drug substance was formulated in 4.59 mg/mL Histidine (3.5% wt), 4.28 mg/mL Histidine HCl (3.3% wt), 26.33 mg/mL Arginine HCl (20.2% wt) and 0.5% polysorbate 80 adjusted to pH 6.3. MLN0002 reference standard was formulated in the same solution described.

In the first step, the solutions required to run the HPLC were prepared. The buffer used contained 100 mM of sodium phosphate (J. T Baker, No. 4011-1, ACS Grade and No. 4062-1, Ultra-pure Bioreagent), 300 mM of sodium chloride (J. T Baker, Cat No. JT3627-5), 10% v/v (EM Science, No. AX0155-1, HPLC Grade), adjusted to a pH range of 6.7-6.9 using 10 M sodium hydroxide (VWR, No. 3247-4) or 1 M hydrochloric acid (VWR, No. 3202-1). Stock solution of GFS (Bio-Rad, Cat. #151-1901) was made by adding 500 μL of 1×PBS (Gibco, catalog #: 186000384c) to a vial of lyophilized GFS and vortexed repeatedly. 75 μL aliquots were stored at −20° C. until needed. The reconstituted GFS standard was kept at −20° C. for up to one month. GFS working solution was prepared by thawing one tube of prepared GFS stock solution and diluted1:4 with 1×PBS and mixed well. The column (Tosoh Biosep, TSK Gel, G3000 SWXL, Cat #08541, 7.8 mm×30 cm, 5 mm particle size) used for analysis was pre-equilibrated. The HPLC system pressure (with the guard column (Tosoh Biosep, TSK Gel, Guard SWXL, Cat #08543, 6.0 mm×4 cm, 7 mm particle size) and column attached) did not exceed 69 bar (1000 psi). Finally, the needle wash contained 10% CAN, the column temperature was set to 25.0° C. and the wavelength on the PDA detector was set to 280 nm.

First, the flow was set for 0.1 mL/min, then increased by 0.1 mL/min about every 10 minutes (allowing the pressure to stabilize prior to increase), until the target flow rate of 0.5 mL/min was reached. The column was equilibrated for a minimum of 115 minutes at a flow rate of 0.5 mL/min prior to analysis.

For samples and standards analysis, the HPLC parameters were set as follows: pump mode was set on isocratic, flow rate of 0.5 mL/min (100% solvent A—the buffer), each sample run for 35 min, 100 μL injections, column temperature 25° C.±3° C. while samples kept at temperature of 4° C.±3° C.

### 1.2 Results

1.2.1 Results from In Vitro Release Experiments of Formulations F1 to F51 Containing Non-Dialyzed Drug

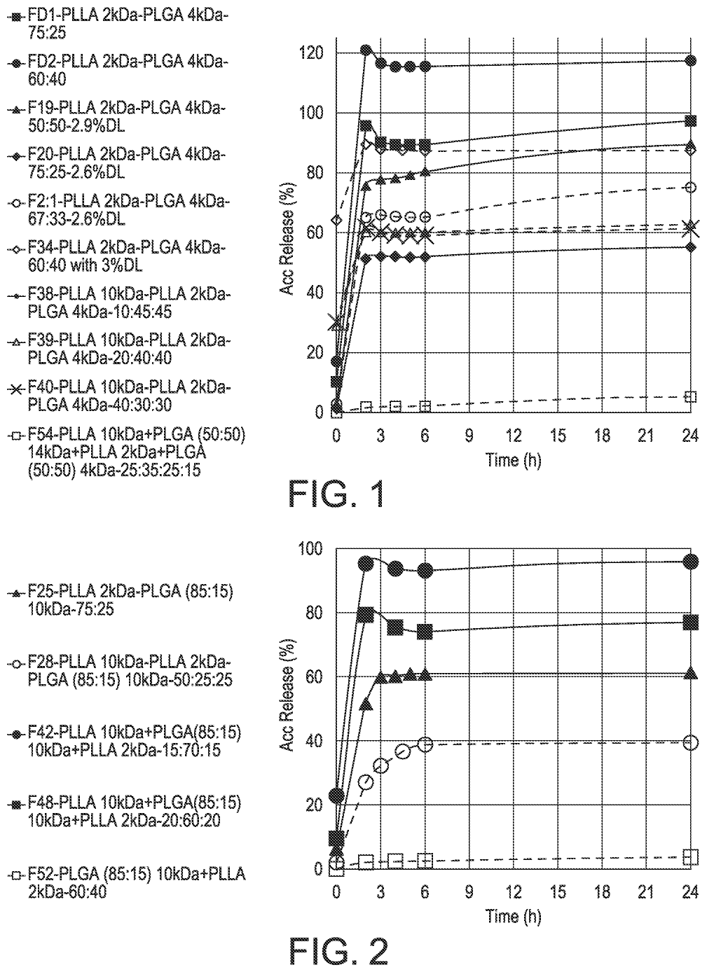

FIG. 1 shows the release profile data. Percent release of encapsulated drug over time (˜24 hours) for formulations FD1, FD2, F19, F20, F21, F34, F38, F39, F40 containing non-dialyzed drug and F54 containing the dialyzed drug.

FIG. 2 shows the release profile data. Percent release of encapsulated drug over time (˜24 hours) for formulations F25, F28, F42 and F48 containing non-dialyzed drug and F52 containing the dialyzed drug.

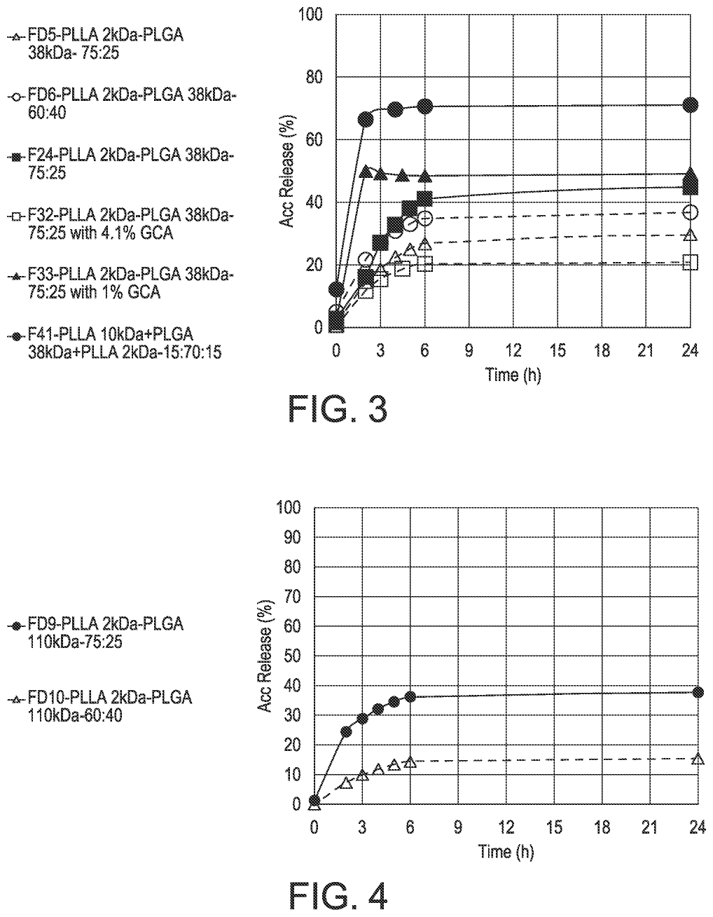

FIG. 3 shows the percent release of encapsulated drug over time (˜24 hours) for formulations FD5, FD6, F24 and F32, F33, and F41 containing non-dialyzed drug.

FIG. 4 shows the percent release of encapsulated drug over time (˜24 hours) for formulations FD9 and FD10, containing non-dialyzed drug.

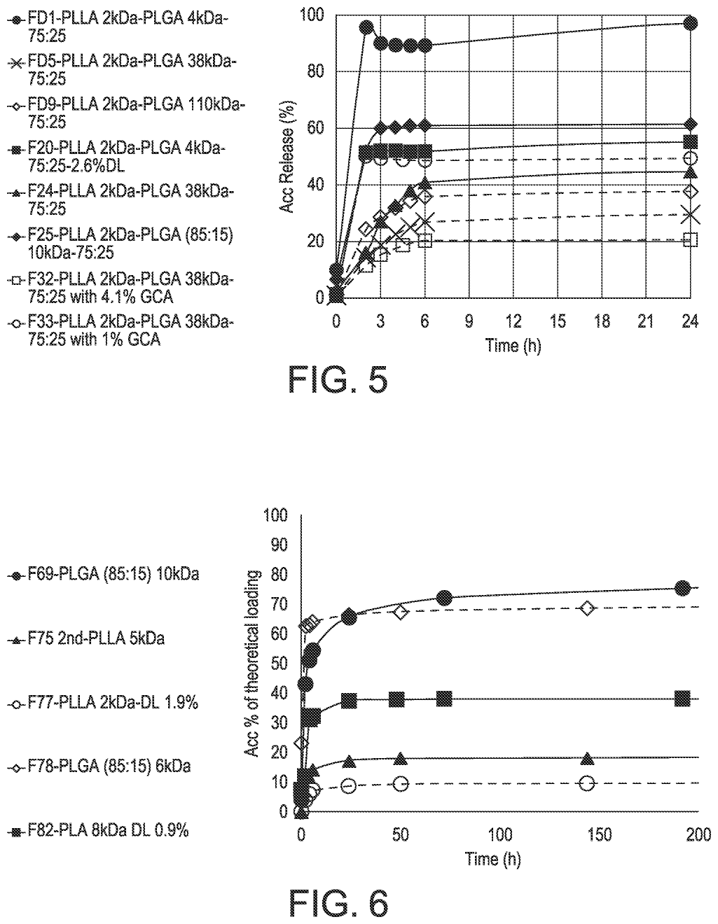

FIG. 5 shows the percent release of encapsulated drug over time (˜24 hours) for formulations FD1, FD5, FD9, F20, F24, F25, F32, and F33 containing non-dialyzed drug.

1.2.2 Results from In Vitro Release Experiments of Formulations F52 to F83 Containing the Dialyzed Drug

A series of in vitro measurements were taken to determine the release profile of the nanoparticle formulations, ranging from a zero time point and up to 1 week. For some formulations (namely, F52H, F53H, and F54H), measurements continued to be taken for up to 2,000 hours, and for a few formulations (namely, F52, F53, and F54) measurements continued to be taken for up to 3,000 hours.

Generally, the drug encapsulation efficiency was at least 80% and the amount of drug released in a week varied from a total of 5% to 100% of the encapsulated drug. In vitro experiments of Formulations F52-F83 demonstrated different patterns for their release profiles over time. Most of the encapsulated drug release was observed between 4 and 200 hours. No additional appreciable release was observed for the formulations that were tested between 200 and 3,000 hours. However, as only 50-60% of the theoretical loading was observed to be released, it is believed that additional drug remained encapsulated in the polymer and some or all the remaining encapsulated drug could be released after 3,000 hours.

All the tested formulations showed a burst release of 30% or less of the theoretical loading amounts at time 0 hour. Most of the tested formulations showed a burst release of 10% or less of the theoretical loading amounts at time 0 hour. However, the following formulations showed a burst release of greater than 10% at time 0 hour: F59, F61, F62 F68, and F78.

### 1.2.2.1 Results of In Vitro Experiments for Drug Containing One Polymer or One Copolymer

The in vitro release data for formulations containing only a single type of polymer reveal that PLLAs of all molecular weights tested released less than 10% of the encapsulated micronized antibody at time 0 hour, i.e., they did not have burst release of the antibody. Some of these formulations even showed no initial drug release, i.e., no drug was released at time 0 hour (see F77 and F75 in FIG. 6).

The higher the molecular weight of the PLA polymer, the more total drug was released while preventing burst release (i.e., less than 10% of the drug released at 0 hour). For example, F75 contained PLLA 5 kDa and had the same release profile as F77, which contained PLLA 2 kDa. F75 released 12-15% of the drug and F77 released ˜10% of the drug over 1 week (FIG. 6). As demonstrated by the release data associated with formulation F82, D, L PLA 8 kDa has no burst release of the encapsulated antibody and provides greater release of the antibody within the first 4 hours compared to formulations F75 and F77, which contained PLA at lower molecular weights (see FIG. 6). Formulations F78 and F69, which contained PLGA 85:15 at molecular weights 6 and 10 kDa, increased the amount of antibody released over the one-week period (see FIG. 6). However, size of the PLGA polymer affects the burst release of the drug with lower molecular weights of PLGA 85:15 polymers, such as 6 kDa or less, exhibiting burst release. For example, F78 contained PLGA 85:15 (6 kDa) and had ˜22% of the drug released at time Ohr while F69 contained PLGA 85:15 (10 kDa) and had <10% of the drug released at time 0 hour (FIG. 6).

These results show that nanoparticles formed of a single polymer or a single copolymer (i.e., PLA or PLGA) having a molecular weight of between 6 kDa and 10 kDa have reduced burst effect at 0 hour. Selection of molecular weight, polymer, and drug loading can be used to modulate release dynamics beginning after 0 hour.

### 1.2.2.2 Results of In Vitro Experiments for Antibody Containing Multiple Polymers or Copolymers

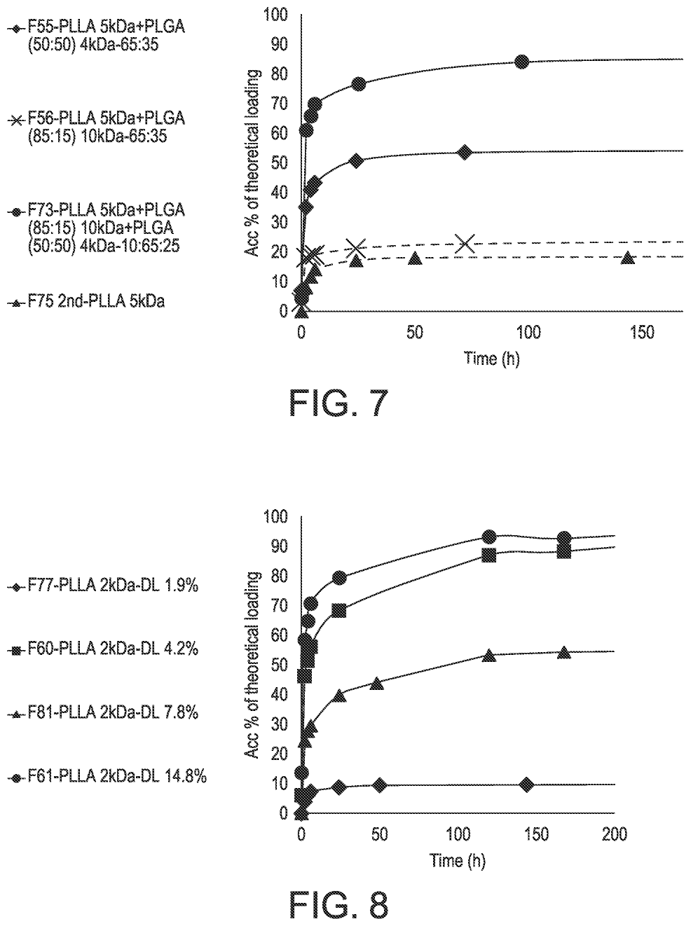

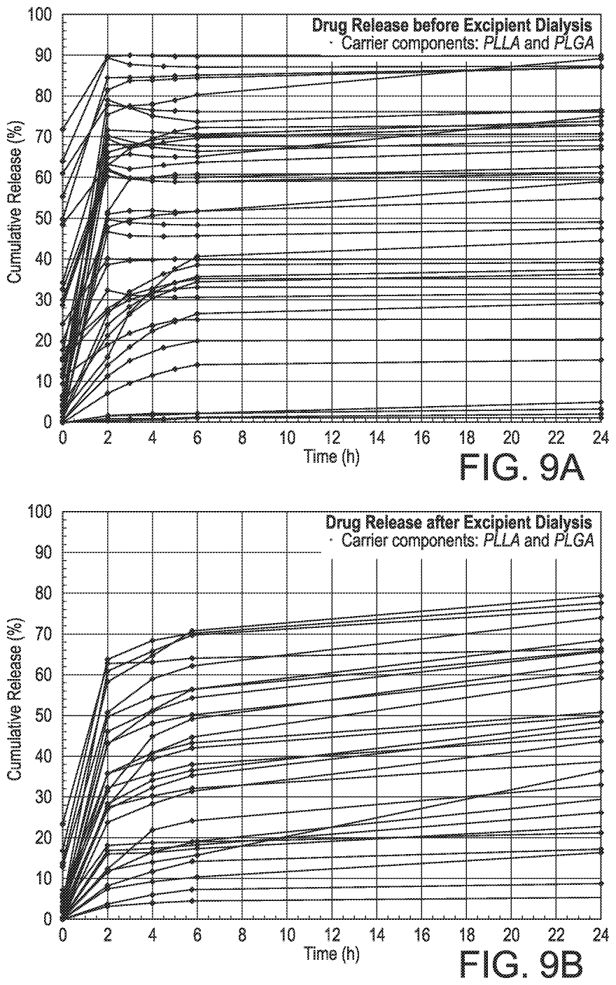

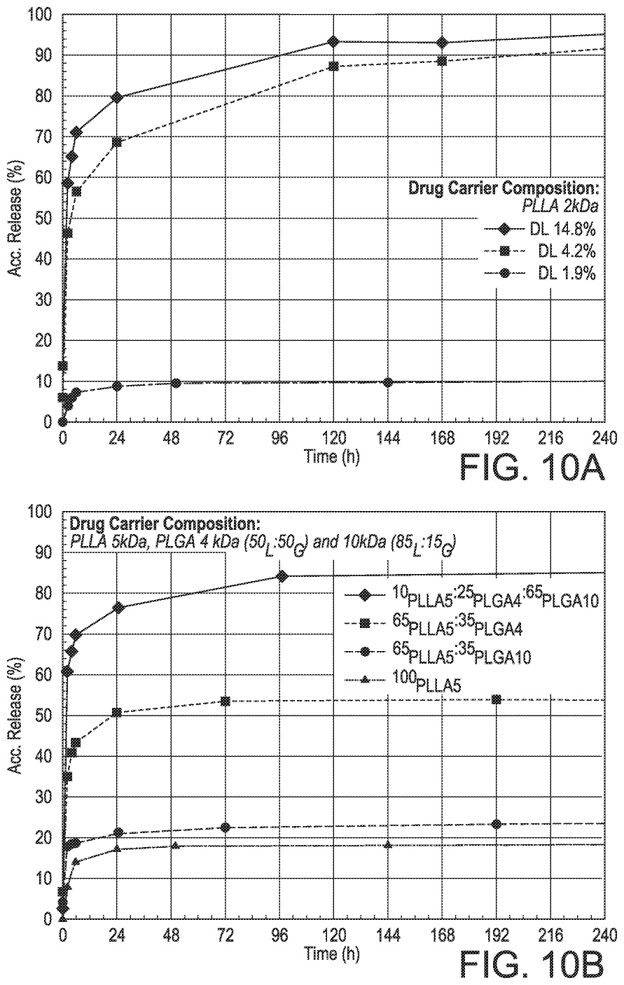

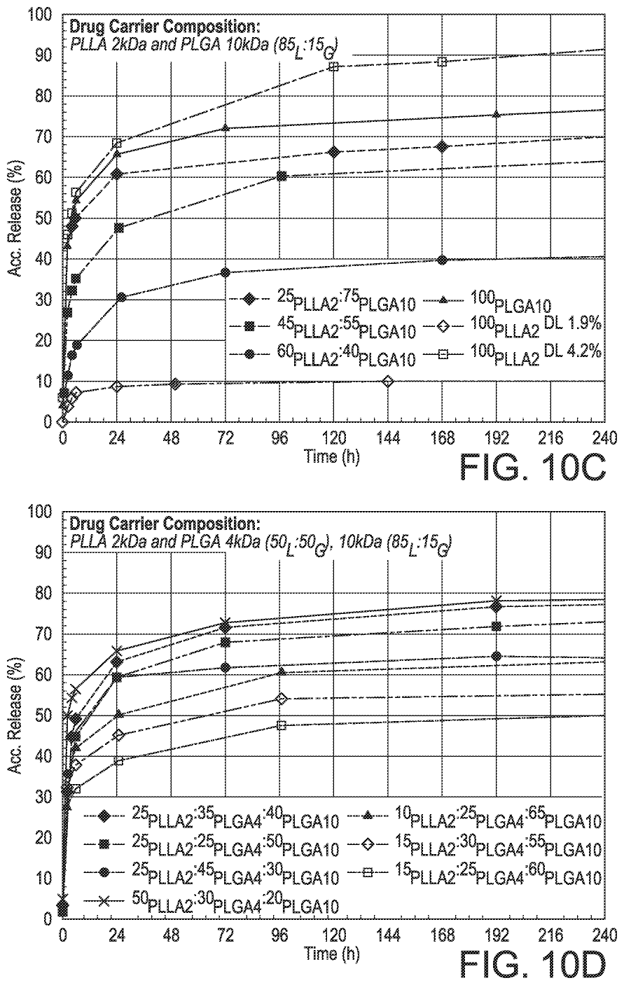

FIG. 7 shows the release profiles of exemplary formulations containing mixtures of polymers. As mentioned, antibody encapsuled in PLLA polymers alone prevents initial burst release of the drug and has lower amounts of total drug released over time compared to antibody encapsulated in PLGA polymers (see FIG. 6). Combining a PLLA polymer with a PLGA polymer was observed to increase the amount of drug released over time without increasing the burst release of the drug, i.e., amount of drug released at time 0 hr. For example, formulations containing a mixture of PLLA (MW 5 kDa) and PLGA 85:15 (10 kDa) or PLGA (50:50) has no burst release and released greater amounts of drug over 1 week compared to F75, which contain PLLA (5 kDa), but did not contain any PLGA polymer (see FIG. 7). For example, F56 contained PLLA (MW 5 kDa) and PLGA 85:15 (10 kDa) in a ratio of 65:35 PLLA to PLGA and resulted in a slight increase in the amount of drug over time compared to F75. formulation F55 contained PLLA (5 kDa) and PLGA 50:50 (4 kDa) in a ratio of 65:35 PLLA to PLGA further increased the amount of drug released in a week. Finally, F73, contained PLLA (5 kDa) and two PLGA polymers, i.e., PLGA 85:15 (10 kDa) and PLGA 50:50 (4 kDa) in a ratio of 10:65:25 of PLLA to PLGA 85:15 to PLGA 50:50 increased the amount of drug released in one week. In sum, combinations of PLA and PLGA at different weights and ratios can also be used to modulate release without increasing burst release.

### 1.2.2.3 Effect of Drug Loading on Release Profile of Formulations Containing Only a Single Polymer or Copolymer

FIG. 8. shows representative data illustrating the effects of varying drug doses on the release profile of formulations incorporating the PLLA 2 kDa polymer. Loading small drug doses into the encapsulating PLLA polymer results in lower release of the antibody over a one-week period compared to higher doses of encapsulated antibody. For example, F60 and F81 containing PLLA (2 kDa) and drug loadings of 4.2% (wt) and 7.8% (wt), respectively, and have greater release over time compared to F77, which contains 1.9% (wt) encapsulated antibody (FIG. 8). However, increasing drug dose to drug loadings greater than 20% (wt), optionally greater than 15% (wt), results in an increase in burst release, such that the formulations exhibit burst release of greater than 10% at time 0 hr. For example, formulation F61 containing PLLA (2 kDa) 14.8% (wt) of the encapsulated antibody, exhibits a burst release of 15% (see FIG. 8). Thus, to minimize or avoid burst release, the antibody loading in the formulation is preferably 15% wt or less, such as between 0.1 and 15% wt, 0.3% wt and 15% wt, or between about 0.3% wt and 10% wt.

### 1.2.2.4 Effect of Drug Loading on Release Profile of Formulations Containing Combinations of Polymers

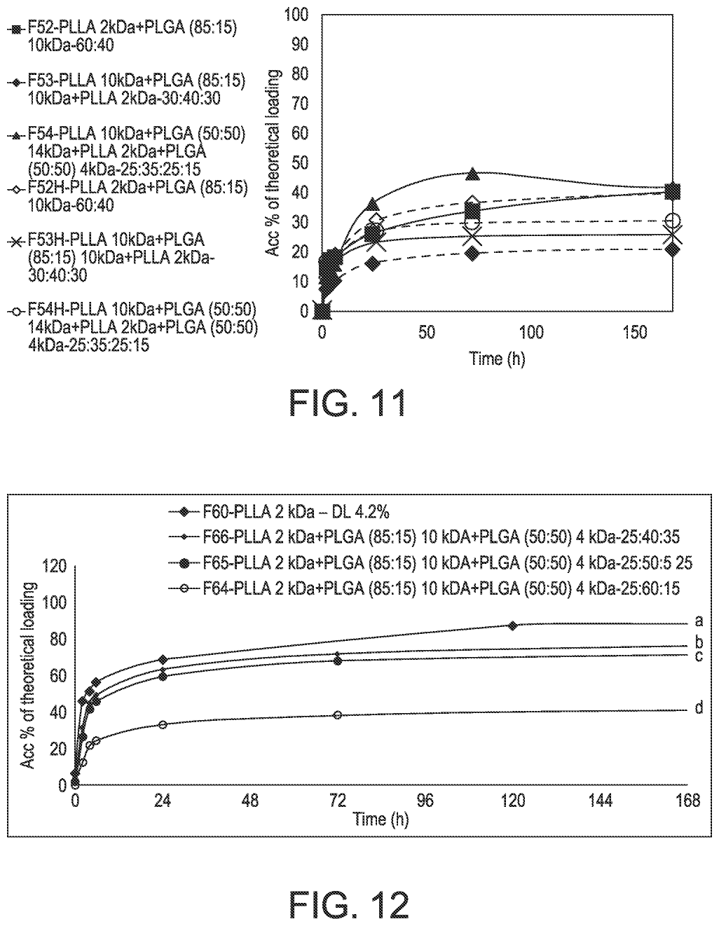

As shown by the data in FIG. 11, different ratios of PLA and PLGA in varying combinations of molecular weights and lactic acid to glycolic acid ratios can be used to control release dynamics while maintaining low initial burst release at 0 hour. For formulations with drug loadings that are less than 10% wt, loading larger drug doses into the encapsulating PLGA and PLLA co-polymer has no effect on the burst release of drug at time point 0 hour and did not have significant changes in the drug release profile thereafter (FIG. 11).

For example, formulations F54 and F54H contain two PLLA polymers (10 kDa and 2 kDa) and two PLGA polymers (85:15 and 50:50) in a ratio of 25% PLLA (10 kDa), 35% PLGA (85:15), 25% PLLA (2 kDa) and 15% PLGA (50:50). F54 and F54H are loaded with 0.3% and 2% of the antibody, respectively, and both formulations exhibit burst release of less than 10% of the drug at 0 hour (FIG. 11). Although the release profile remained the same for both the formulations with low and high doses of drug loading, F54 released more drug over a one-week period compared to F5H (FIG. 11). Another copolymer formulation, F53 contained two PLLA polymers (10 kDa and 2 kDa) and one PLGA polymer 85:15 (10 kDa) in a ratio of 30% PLLA (10 kDa), 40% PLGA (85:15) and 30% PLLA (2 kDa) (FIG. 11). Both F53 and F53H have less than 10% burst release at 0 hour and similar release profiles thereafter. However, formulation F53 released less drug over time compared to F53H with higher loading of drug (2% wt) (FIG. 11). Altogether, combinations of PLLA and different PLGA polymers in varying ratios and molecular weights can be used to deliver different doses of drug, where the drug loading is less than 10% wt, while maintaining low burst release of the drug at time 0 hour.

### 1.2.2.5 24 Hour In Vitro Release of Exemplary Formulations

The release in a 24-hour period can be divided into two phases.

In the first release phase, an initial bolus of the antibody is released during the first 4 hours, i.e., from the zero time point until 4 hours. Table 3 shows representative formulations for drug release within 4 hours from the zero time point, with the acceptable upper limit for 4 hours drug release being less than 30% (wt) of the theoretical amount of antibody. All the formulations listed in Table 3 had <10% (wt) burst release of the antibody at time 0 hour and are made from a single polymer or a mixture of polymers. For example, Formulation F81 contained only a single encapsulating polymer, i.e., PLLA (2 kDa), and released 23% of the micronized drug within 4 hours (see Table 3). F54 contained a mixture of two polymers, PLLA1 (5 kDa) and PLGA 85:15 (10 kDa) in a ratio of 65:35 PLLAI to PLGA and released 14% of the micronized drug within 4 hours (Table 1). Formulation F73 contained a mixture of three polymers, i.e. PLLA (2 kDa), PLGA (10 kDa), and PLLA (4 kDa) in a ratio of 10:65:25 PLLA:PLGA:PLLA, and released 61% of the drug within 4 hours. Formulations, such as those listed in Table 3, which demonstrated a low burst release at time 0 hour and with 30% wt or less of the antibody released by 4 hours, are preferred for injectable methods of administration, such as subcutaneous or intraperitoneal delivery.

TABLE 3
Representative formulations without burst release and releasing
up to 30% (wt) antibody within 4 hours (i.e., during phase 1).

| Formulation | At 0 | Δ0-4 h | Δ4-24 h | Total for 24 h |
|---|---|---|---|---|
| F76 | 0 | 4 | 1 | 5 |
| F77 | 0 | 6 | 3 | 9 |
| F53 | 0 | 9 | 7 | 16 |
| F75 | 0 | 12 | 6 | 17 |
| F54 | 0 | 14 | 22 | 36 |
| F53H | 1 | 16 | 7 | 23 |
| F56 | 3 | 16 | 3 | 21 |
| F52H | 0 | 16 | 14 | 31 |
| F54H | 0 | 17 | 10 | 27 |
| F52 | 0 | 17 | 9 | 26 |
| F64 | 0 | 22 | 11 | 33 |
| F81 | 0 | 23 | 14 | 37 |
| F82 | 7 | 24 | 6 | 37 |
| F70 | 2 | 28 | 9 | 39 |
| F79 | 0 | 28 | 15 | 44 |
| F83 | 2 | 29 | 8 | 38 |
| F71 | 6 | 30 | 10 | 45 |

In the second release phase, a second bolus of antibody was released, i.e., between 4 hours and 24 hours. Tables 4 and 5 lists the representative formulations for drug release between 4 and 24 hours. The amount of antibody released between 4 and 24 hours ranged from 10-22% (wt) of theoretical drug load. All the formulations listed in Table 4 had <10% (wt) burst release of the antibody at time 0 hour except F61, which contained only the PLLA1 2 kDa polymer, and formulation F68, which contained a mixture of the PLGA 85:15 (10 kDa) and the PLLA2 4 kDa polymers in a ratio of 60:40 PLGA to PLLA2. Formulations F54, 567, F66, F80, F65 and F60 had the most desirable profiles for modulated release of the micronized antibody between 4 and 24 hours (Table 4). Therefore, formulations with low burst release at time 0 hours, but 30% or less release by 4 hours are preferred for oral methods of administration, because this initial delay allows the composition to traverse the upper gastrointestinal tract and arrive at the intended delivery location prior to the bulk of drug release. The micronized antibody was released in higher amounts at 24 hours from the following formulations: F62, F61, F59, F73, F68, and F60 compared to the other tested formulations, albeit, with burst release of the drug exceeding 10% at 0 hour and more than 30% of the drug released between 0-4 h (Table 3).

Preferred oral formulations release X %-Y % of drug between 4 and 24 hours. In some preferred injectable and/or oral delivery formulations, release continues during 4-24 and/or after 24 hours. For example, formulations F60 released ˜20% of the encapsulated drug after 24 hours. Other exemplary formulations include F64, F65 and F66 which had ˜7-9% of the encapsulated drug released after 24 hours.

TABLE 4
Representative Formulations organized by greatest
release during 2^{nd} phase (i.e., from 4 h to 24 h)

| Formulation | At 0 | Δ0-4 h | Δ4-24 h | Total for 24 h |
|---|---|---|---|---|
| F54 | 0 | 14 | 22 | 36 |
| F67 | 2 | 38 | 18 | 59 |
| F66 | 4 | 41 | 18 | 63 |
| F80 | 0 | 44 | 18 | 62 |
| F65 | 2 | 40 | 18 | 59 |
| F60 | 6 | 45 | 17 | 68 |
| F74 | 0 | 32 | 15 | 48 |
| F79 | 0 | 28 | 15 | 44 |
| F68 | 13 | 46 | 15 | 74 |
| F69 | 4 | 47 | 15 | 66 |
| F61 | 14 | 51 | 15 | 79 |
| F81 | 0 | 23 | 14 | 37 |
| F57 | 0 | 34 | 14 | 48 |
| F52H | 0 | 16 | 14 | 31 |
| F63 | 0 | 48 | 13 | 61 |
| F58 | 5 | 49 | 11 | 66 |
| F64 | 0 | 22 | 11 | 33 |
| F72 | 3 | 36 | 11 | 50 |
| F73 | 4 | 61 | 11 | 77 |
| F55 | 7 | 34 | 10 | 51 |
| F54H | 0 | 17 | 10 | 27 |
| F71 | 6 | 30 | 10 | 45 |

TABLE 5
Representative Formulations organized by greatest
release during 2^{nd} phase (i.e., from 4 h to 24 h)

| Formulation | At 0 | Δ0-4 h | Δ4-24 h | Total for 24 h |
|---|---|---|---|---|
| F62 | 27 | 65 | 9 | 102 |
| F61 | 14 | 51 | 15 | 79 |
| F59 | 17 | 51 | 9 | 78 |
| F73 | 4 | 61 | 11 | 77 |
| F68 | 13 | 46 | 15 | 74 |
| F60 | 6 | 45 | 17 | 68 |
| F78 | 23 | 40 | 3 | 66 |
| F58 | 5 | 49 | 11 | 66 |
| F69 | 4 | 47 | 15 | 66 |
| F66 | 4 | 41 | 18 | 63 |
| F80 | 0 | 44 | 18 | 62 |
| F63 | 0 | 48 | 13 | 61 |
| F65 | 2 | 40 | 18 | 59 |
| F67 | 2 | 38 | 18 | 59 |
| F55 | 7 | 34 | 10 | 51 |
| F72 | 3 | 36 | 11 | 50 |
| F57 | 0 | 34 | 14 | 48 |
| F74 | 0 | 32 | 15 | 48 |

### Example 2: In Vivo Release Experiments Dialyzed Antibody

### 2.1 Materials and Methods

### 2.1.1 Protocol for Isolated Loop (IL) In-Vivo Experiment

Sprague Dawley (SD) rats were used for all in-vivo experiments (average weight of 250 g to 780 g). Rats were fully anesthetized using 1.5-4% isoflurane and kept under anesthesia and on a heating pad set to low throughout the experiment. First, 300 μL of blood was withdrawn via the rat's tail to establish baseline concentration (zero-point concentration). Then the rat's abdomen region was hair clipped and the skin was cleaned with alternating betadine and alcohol three times. Next, a 2.5 cm incision was made on the ventral midline of the abdomen using a #15 scalpel blade. 10-20 cm loop of the jejunum (or the relevant GI section) was gently isolated and exteriorized. One side of the loop was ligated using a 4-0 absorbable suture. The other side was tied loosely and 0.5-1 mL of a solution containing relevant formulation, i.e., F65N or F82, was injected using a 21G needle next to the loose end. Once the injection was complete, the thread was tightened, thereby isolating the loop from the rest of the GI. The isolated loop (IL) was then placed back into the abdominal cavity of the rat and washed with warm sterile saline prior to incision closure. The incision was sealed with 4-0 absorbable sutures in a simple continuous pattern. Blood samples were collected via the rat's tail only at the zero time point and 5 hours post injection.

After 5 hours, the rat was euthanized using a CO₂ chamber and by puncturing its diaphragm. Then the IL was washed three times by running 2 mL of saline through it. The washout solution was collected for IL content analysis. The IL tissue was also harvested for IL tissue content to analyze whether local delivery of the antibody occurred. The tissue and washout solution were kept at −80° C. until analyzed. The blood samples were left for 25 minutes at room temperature and centrifuged for 18 minutes at 4 0° C. and under 2000 g. Serum was collected and the samples were kept in −80° C. until analyzed.

### 2.1.2 IL Experiments for Formulations F82 and F65N

As mentioned in Example 1, formulations of interest demonstrated maximum release within 24 to 48 hours or sustained release over a one-week period while demonstrating a burst release of less than 10% of the encapsulated micronized drug at the zero time point.

To test whether such formulations are useful for local delivery of an encapsulated antibody, two formulations were selected for IL experiments, F82 and F65N. Formulation F82 contained only D, L PLA (8 kDA) and was loaded with 0.9% of the micronized antibody Formulation F82 was dissolved in 1.17 mL DI water rather than the Takeda Dispersion Buffer to “induce” the right charge.

Takeda Dispersion Buffer is a solution of pH 4.0 containing 75 mM of citric acid buffer, 5% (w/v) mannitol, 1% (w/v) Poly vinyl-pyrrolidone (PVP, 10 kDa), and 0.05% polysorbate 80 to hinder enzymatic degradation of the antibody in the GI.

Two male Sprague Dawley rats weighing 640 g (Rat #1) and 595 g (Rat #2) received an injection into a 10 cm or 20 cm IL of the jejunum and were monitored for 5 hours. Rat #1 received an injection of 1 mL 27.7 mg F82, and Rat #2 received an injection of 0.8-0.85 mL 22-23.4 mg F82.

Formulation F65N was made of PLLA (2 kDa) and two PLGA polymers, PLGA1 85:15 (10 kDa) and PLGA: 50:50 (4 kDa) in a weight ratio of 25:50:25 PLLA:PLGA1:PLGA2. 23.1 mg of Formulation F65N was dispersed in 1.26-1.29 mL Takeda Dispersion Buffer. Two male Sprague Dawley rats weighing 581 (Rat #3) and 610 g (Rat #4) received an injection respectively into a 10 cm IL of the jejunum and were monitored for 5 hours. Rat #3 received an injection of 0.85-0.9 mL 15.6-16.5 mg F65N, and Rat #4 received an injection of 0.9-0.95 mL 16.1-17.0 mg F65N.

### 2.1.3 Protocol for the Intravenous (IV) Drug Delivery

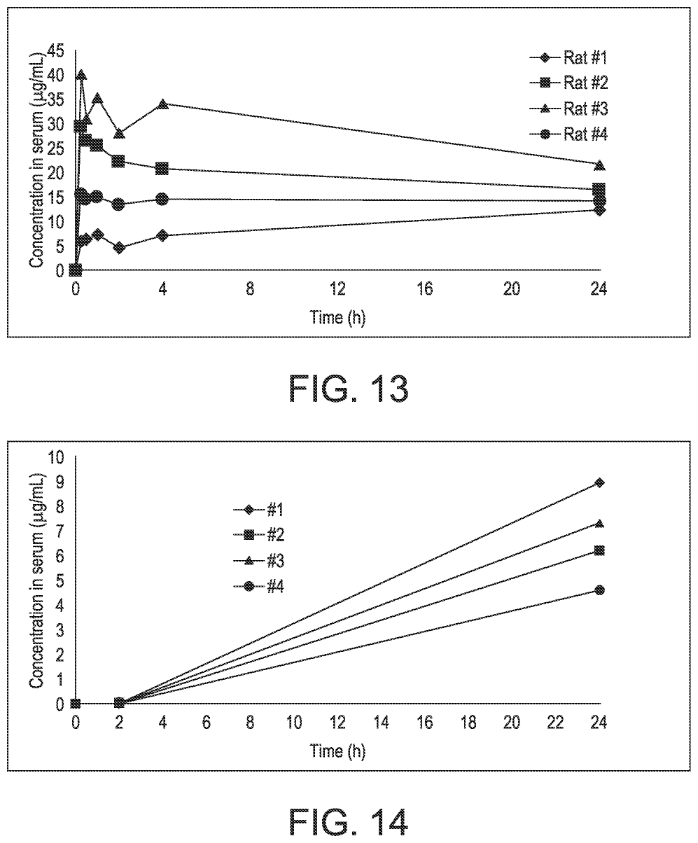

Four Sprague-Dawley rats, weighing at least 250 g were anesthetized using 1.5-4% isoflurane and kept under anesthesia on a heating pad set to low throughout the experiment 300 μL of blood was withdrawn via the rat's tail to establish a baseline concentration (See 0-hour, FIG. 13). The rats were then given an intravenous injection of 0.75 mg pure antibody in water Blood samples (˜300 μL) were withdrawn via the rat's tail at 1-, 2-, 3-, 4-, 5- and 24-hours post-injection. The blood samples were left for 25 minutes at room temperature and centrifuged for 18 minutes at 4° C. and under 2000 g. Serum was collected and the samples were kept in −80° C. until analyzed. At the end of the experiment, the rat was euthanized in a CO₂ chamber and by puncturing its diaphragm.

FIG. 13 shows the serum concentrations (μL/mL) detected over time following intravenous injection of the antibody.

Bioavailability (BA) percentage was calculated by dividing the area under the curve (AUC) for IL experiments by the AUC for an IV injection for the same time point (0 hour to 5 hours) and dosage per rat (0.75 mg of antibody per rat) and multiplying by 100.

### 2.1.4 Protocol for Subcutaneous Drug Delivery

Four Sprague Dawley rats weighing 250 g to 400 g were used for all in vivo experiments. Rats were fully anesthetized using isoflurane and kept under anesthesia and on a heating pad set to low throughout the injection part of the experiment. First, 300 μL of blood was withdrawn via the rat's tail to establish baseline concentration. Then 1 mL of solution was injected subcutaneously into the skin along the middle of the rat's back using a 21G needle. Blood samples were drawn via the rat's tail 0-4- and 24-hours post injection. The blood samples were left for 25 minutes at room temperature and centrifuged for 18 minutes at 4° C. and under 2000 g. Serum was collected and the samples were kept in −80° C. until analyzed. At the end of the experiment, the rat was euthanized using a CO₂ chamber and by puncturing its diaphragm.

FIG. 14 shows the serum concentrations (μL/mL) detected over time following subcutaneous injection of the antibody.

Bioavailability (BA) percentage was calculated by comparing the area under the curve (AUC) for IL experiments with the AUC for a subcutaneous injection for the same time point (for example, 0 hour to 5 hours) and dosage per rat (0.75 mg of antibody). The average bioavailability was determined as explained above.

### 2.2 Results

The data in Table 6 compares the bioavailability by direct injection to the GI tract by isolated loop (IL) of the selected formulations, F82 and F65N, administered in the IL experiments five hours after administration, with the bioavailabilities for the same formulations administered via direct intravenous (IV) and subcutaneous (SQ) injections. The total drug dose per rat for the IL experiments was approximately 0.375 mg per rat, which is about 50% less than the dose administered in the IV and SQ injection experiments in which the rats received a dose of about 0.75 mg per

The bioavailability (BA) was calculated using a single point comparison i.e., comparing the average concentration in the blood after 5 hr. The rat was first injected with an IV dose of 0.75 mg antibody and the concentration in the blood was measured over 24 hours, (see FIG. 13). Next, the rat was given a 0.75 mg subcutaneous injection of the antibody and the concentration of the antibody in the blood was measured at intervals over 24 hours (see FIG. 14).

The concentration of the antibody in the blood was measured every hour for 5 hours for each rat given the encapsuled drug in microspheres via the isolated loop protocol.

For the isolated loop (IL) experiments, the area under the curve (AUC) was determined for 5 hours and divided by the corresponding AUC determined from the IV graph, taking the AUC only after 5 hours. The same calculation was performed for the SQ data, finding the AUC for each rat that was administered the Intraluminal microsphere drug, and dividing the result by the corresponding AUC of the SQ curve taken at 5 hours. Given that the drug doses of IV, SQ and IL are the same, relative bioavailability was then calculated as a ratio of the IL to IV BA or IL to SQ BA at 5 hours.

Table 6 represents the relative bioavailability for the isolated loop experiments. Four animals were used for the study and relative bioavailability was found to be 3.0% as compared to IV injection and 73% compared to SQ injection.

TABLE 6
Percent bioavailability from IL experiments for formulations
F82 and F65N compared to the average bioavailability
for IV and SQ administration in rats 3.0% as compared
to IV injection and 73% compared to SQ injection.

| | BA (%) |
|---|---|
| To IV | |
| Rat #1 (F82) | 3.7 |
| Rat #2 (F82) | 2.0 |
| Rat #3 (F65N) | 4.3 |
| Rat #4 (F65N) | 2.4 |
| AVG BA: | 3.08 |
| To SQ | |
| Rat #1 (F82) | 86.8 |
| Rat #2 (F82) | 47.0 |
| Rat #3 (F65N) | 102.2 |
| Rat #4 (F65N) | 56.6 |
| AVG BA: | 73.2 |

As shown in Table 6, the relative bioavailability as compared to IV drug administration is only ˜3%. Theoretically, the bioavailability will be higher if the isolated loop experiments could be extended beyond the 5-hour time limit. For this reason, the results comparing the bioavailability following the IL injections to that of the SQ injections better demonstrates the biological activity of the formulation and the relative bioavailability. The bioavailability following IL injections of the encapsulated drug containing formulations F82 and F65N was higher compared to the bioavailability following the SQ injections of the same formulations (See Table 6). For example, the bioavailability of the F82 formulation in Rats #1 and #2 in the IL experiments were 86.8% and 47.0%, respectively, of the bioavailability observed in the SQ injection experiments (see Table 6). Also, in Rat #3, the encapsulated antibody in formulation F65N had a higher bioavailability following the SQ injection compared to the IL injection of formulation F65N (see Rat #3, Table 6).

1. A pharmaceutical composition for delivering an antibody comprising a micronized antibody having a molecular weight of 100 kDa or greater and a size in the range of at least 20 nm and less than 600 nm encapsulated or dispersed in a microparticle, a nanoparticle, or a combination thereof having a diameter in the range of about 0.3 μm to about 2 μm, and comprising one or more polyhydroxyester polymers having an average molecular weight of between about 2 kDa and about 30 kDa, inclusive,
wherein the composition provides sustained release of the micronized antibody with less than 10% of the micronized antibody released initially (0 hour).

2-4. (canceled)

5. The pharmaceutical composition of claim 1, wherein the micronized antibody has a loading in the microparticles and/or nanoparticles between 0.3% w/w and 20% w/w, optionally between 0.3% w/w and 15% w/w, optionally between 0.3% w/w and 10% w/w.

6. The pharmaceutical composition of claim 1, wherein the micronized antibody is released for at least 4 hours, at least 12 hours, at least 24 hours, at least 36 hours, at least 48 hours, up to 100 hours, up to 200 hours, or up to 300 hours or longer in vitro or following administration.

7. The pharmaceutical composition of claim 1 comprising at least one polyhdroxyester polymer selected from poly-L-lactide (PLLA) having an average molecular weight of about 10 kDa or less, poly-D-lactide (PDLA) having an average molecular weight in the range of about 2 to about 14 kDa, poly-D,L-lactide, and poly(lactic-co-glycolic acid) (PLGA) having an average molecular weight range of about 1 kDa to about 120 kDa, or comprising a blend of two or more of the polyhdroxyester polymers.

8-14. (canceled)

15. The pharmaceutical composition of claim 1, wherein the micronized antibody comprises less than 50% w/w, less than 40% w/w, less than 30% w/w, less than 20% w/w, less than 10% w/w, or less than 5% w/w antibody stabilizing excipients having a molecular weight of less than 20 kDa, preferably wherein the antibody stabilizing excipients are non-polymeric.

16. The pharmaceutical composition of claim 1, wherein the antibody is dialyzed via 20 kDa molecular weight cut off dialysis cassette for at least 1 min, such as for about 2 hours or about 24 hours, prior to or post-micronization.

17. (canceled)

18. The pharmaceutical composition of claim 1, wherein the microparticle, nanoparticle, or combination thereof is formed of a blend comprising two or more polyhydroxyester polymers, and wherein the blend comprises (i) a polyhydroxyester homopolymer and a polyhydroxyester co-polymer (such as PLGA, PDLLA, etc) or (ii) two or more polyhydroxyester homopolymers, such as a blend between PLLA and PLDA.

19-20. (canceled)

21. The pharmaceutical composition of claim 1, wherein the microparticle, nanoparticle, or combination thereof comprises a blend comprising at least two of the polyhydroxyester polymers (i), (ii), and (iii),
(i) 1-30% w/w PLLA of 2-10 kDa;
(ii) 1-50% w/w PDLA of 2-10 kDa; and
(iii) 70-96% w PLGA of 2-60 kDa; and further comprises
(iv) 4-20% w/w micronized antibody loading,
preferably wherein 80-100% of the micronized antibody is released by 24 hours in medium in vitro.

22. The pharmaceutical composition claim 1, wherein the microparticle, nanoparticle, or combination thereof comprises a blend comprising at least two of the polyhydroxyester polymers (i), (ii), (iii), and (iv),
(i) 1-50% w/w PLLA of 2-10 kDa;
(ii) 1-50% w/w PDLA of 2-10 kDa
(iii) 1-70% w/w PLGA (85:15) of 2-60 kDa;
(iv) 0-30% w/w PLGA (50:50) of 2-60 kDa; and further comprises
(v) up to 15% w/w the micronized antibody loading,
preferably wherein 40-100% of the micronized antibody is released by 200 hours in medium in vitro.

23. The pharmaceutical composition of claim 15 for subcutaneous or intraperitoneal injection, wherein the microparticle, nanoparticle, or combination thereof comprises:
(i) 50-100% w/w PLLA of 2-20 kDa;
(ii) 0-35% w/w PLGA (85:15) of 10-50 kDa; and
(iii) up to 15% w/w of the micronized antibody loading,
preferably, wherein 40-100% of the micronized antibody is released by one month in medium in vitro.

24. The pharmaceutical composition of claim 1, wherein the bioavailability of the formulation at 5 hours following oral delivery is at least 40% of the bioavailability of a subcutaneously administered formulation containing the same antibody unencapsulated in an aqueous solution containing stabilizer for the antibody.

25. The pharmaceutical composition of claim 1, wherein the particle, nanoparticle, or combination thereof further comprises a bioadhesive coating on the surface of the microparticle and/or nanoparticle.

26-31. (canceled)

32. The pharmaceutical composition of claim 1, wherein the microparticle, nanoparticle, or combination thereof comprises 4-20% w/w micronized antibody loading, and the microparticle, nanoparticle, or combination thereof is formed from a mixture comprising at least two of the polyhydroxyester polymers (i), (ii), and (iii) having the following polymer weight ratios between the polyhydroxyester polymers:
(i) 1-30% w/w PLLA of 2-10 kDa;
(ii) 1-50% w/w PDLA of 2-10 kDa; and
(iii) 70-96% w PLGA of 2-60 kDa.

33. (canceled)

34. The pharmaceutical composition of claim 15 for subcutaneous or intraperitoneal injection, wherein the microparticle, nanoparticle, or combination thereof comprises up to 15% w/w micronized antibody loading, and the microparticle, nanoparticle, or combination thereof is formed from the polyhydroxyester polymers (i) and (ii) having the following polymer weight ratios between the polyhydroxyester polymers:
(i) 50-100% w/w PLLA of 2-20 kDa; and
(ii) 0-35% w/w PLGA (85:15) of 10-50 kDa.

35-36. (canceled)

37. The pharmaceutical composition of claim 1, wherein the microparticle, nanoparticle, or combination thereof is in a carrier comprising a pharmaceutically acceptable carrier or excipient for oral or parenteral administration, and wherein the carrier or excipient is L-DOPA.

38-39. (canceled)

40. The pharmaceutical composition of claim 1, wherein the antibody has a molecular weight of at least about 150 kDa.

41. The pharmaceutical composition of claim 1, in a form suitable for systemic delivery via oral administration or via subcutaneous administration.

42-43. (canceled)

44. The pharmaceutical composition of claim 1, in a form suitable for systemic delivery via percutaneous administration to the gastrointestinal tract or in a form suitable for local delivery to the gastrointestinal tract via oral administration or percutaneous administration.

45. (canceled)

46. A method of treating a subject in need thereof comprising administering to the subject the pharmaceutical composition of claim 1.

47. (canceled)

48. A method of making a microparticle, a nanoparticle, or a combination thereof, comprising a micronized antibody having a molecular weight of 100 kDa or greater encapsulated or dispersed in the microparticle, the nanoparticle, or combination thereof, the method comprising
mixing the micronized antibody in a solvent comprising at least two of the polyhydroxyester polymers (i), (ii), and (iii):
(i) about 10% w/w to about 80% w/w PLLA of about 1 kDa to about 10 kDa or about 2 kDa to about 10 kDa;
(ii) about 10% w/w to about 80% w/w PDLA of about 1 kDa to about 10 kDa or about 2 kDa to about 10 kDa; and
(iii) about 10% w/w to about 80% w/w PLGA of about 1 kDa to about 120 kDa, to form a solution, suspension, or dispersion, wherein the percent weights of the polymers are the feed weight ratios between the polyhydroxyester polymers.

49-51. (canceled)
